(19) United States
(12) Patent Application Publication
(10) Pub. No.: US 2005/0153306 A1
(43) Pub. Date: Jul. 14, 2005

(54) Fluorogenic enzyme substrates and uses thereof

(75) Inventors: Jennifer L. Harris, San Diego, CA (US); Robert Damoiseaux, Escondido, CA (US); Bradley J. Backes, Chicago, IL (US); Nicolas Winssinger, La Jolla, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: IRM LLC, Hamilton (BM)

(21) Appl. No.: 10/892,402

(22) Filed: Jul. 14, 2004

Related U.S. Application Data
(60) Provisional application No. 60/487,464, filed on Jul. 14, 2003.

(51) Int. Cl. C12Q 1/68; C07K 14/47

(52) U.S. Cl. 435/6; 530/350

(57) ABSTRACT

The present invention provides, inter alia, fluorogenic enzyme substrates, such as fluorogenic polypeptide substrates, libraries of fluorogenic enzyme substrates and methods for assaying for enzymatically active enzymes, such as hydrolases (e.g., proteases), in biological samples.

### CROSS-REFERENCES TO RELATED APPLICATIONS

This application claims benefit of U.S. Provisional Application No. 60/487,464, filed Jul. 14, 2003, which application is incorporated herein by reference for all purposes.

### BACKGROUND OF THE INVENTION

Proteases are enzymes that effect many vital cellular functions by specifically cleaving proteins. Protease themselves are nearly exclusively regulated by posttranslational modifications. The precise and limited action of proteases is a mechanism by which cells regulate many vital events. The completion of the human genome revealed the existence of about 500 proteases and many of these are involved in the regulation of essential cellular processes such as DNA replication, cell-cycle progression, differentiation, migration, morphogenesis, immunity, haemostasis, neuronal outgrowth, and apoptosis (see, Barret et al., HANDBOOK OF PROTEOLYTIC ENZYMES (Academic Press, London, 1998)). Misregulation of proteolytic activity is involved in many pathological situations like neurodegeneration, cardiovascular diseases, arthritis, cancer and infectious diseases (see, Barret et al., supra). Consequently, proteases are an attractive target for drug screening and the monitoring of protease activity can be diagnostic and prognostic of disease states.

A major characteristic of a given protease is its substrate specificity that can range from very broad—as for proteases involved in catabolism—to very narrow—as for proteases involved in the regulation of cellular events. Knowledge of the substrate specificity of proteases can enable their identification in biological samples using their activity towards specific substrates. Ideally, the assay is performed directly on clinical samples. However, there are significant obstacles to screening clinical samples for multiple protease activities such as the limited availability of larger sample amounts.

To address these issues the use of microarray based tools have been developed. One approach relies on the covalent modification of active proteases with PNA encoded small molecule probes (see, Winssinger et al., Proc. Natl. Acad. Sci. USA, 99:11139-11144 (2002)). After the reaction between the probes and proteases, size exclusion purification separates the probes bound to the proteases from unreacted probes, and hybridization of the encoding PNA tag to an oligonucleotide chip allows for the identification of these probes.

Although the microarray based tools developed by Wissinger et al. are invaluable, there still remains a need in the art for methods that simultaneously detect and measure the proteolytic activity of multiple proteases in complex biological samples. Such methods would be particularly useful in dissecting intricate pathways and identifying relevant proteins involved in such pathways for use as drug targets or as diagnostic markers. Quite importantly, the present invention provides such methods and, in addition, compounds useful in carrying out such methods.

### SUMMARY OF THE INVENTION

The present invention provides fluorogenic enzyme substrates (e.g., fluorogenic protease substrates) linked to peptide nucleic acid (PNA) identifier tags, libraries, e.g., microarrays, of such fluorogenic enzyme substrates, and methods of using such fluorogenic enzyme substrates to identify enzymatic activity. Typically, the enzymatic activity (e.g., proteolysis) is measured by the level of fluorescence upon hybridization of the sample to an oligonucleotide microarray. The fluorogenic substrate strategy provided by the present invention is extremely sensitive since the turnover of enzyme, e.g., protease, leads to signal amplification. In addition, the PNA-based methods of the present invention have the advantage that the enzyme activity (e.g., proteolysis) can be carried out in solution. This is important in order to exclude the effects of nonspecific interactions of the enzymes with the surface and offers better control of substrate/analyte concentration. Moreover, the design of the PNA-based methods of the present invention allows for the use of the powerful and economic split-pool (i.e., split and combine) synthesis which is important for the synthesis of large libraries. As such, the present invention provides PNA encoded enzyme substrate libraries that allow for the simultaneous detection and measurement of the enzyme activity of multiple enzymes in complex biological samples. In particular, the present invention provides PNA encoded proteolysis substrate libraries that allow for the simultaneous detection and measurement of the proteolytic activity of multiple proteases in complex biological samples.

In one embodiment, the present invention provide a fluorogenic enzyme substrate comprising: (a) a fluorogenic moiety; (b) an organic moiety covalently attached to the fluorogenic moiety, wherein the organic moiety comprises a cleavage recognition site for an enzyme; and (c) a petido nucleic acid (PNA) identifier tag covalently attached to the fluorogenic moiety, wherein the PNA identifier tag identifies the organic moiety. In one embodiment, the fluorescence of the fluorogenic moiety is quenched, suppressed or attenuated when the organic moiety is covalently attached to the fluorogenic moiety.

Numerous fluorogenic moieties can be used in the fluorogenic enzyme substrates of the present invention. In a preferred embodiment, the fluorogenic moiety is a rhodamine moiety, such as a rhodamine NHS ester. In another preferred embodiment, the fluorogenic moiety is a coumarin moiety, such as 7-amino-4-methylcoumarin (AMC), 7-amino-4-trifluoromethylcoumarin (AFC), 7-amino-4-chloromethylcoumarin (CMAC) and 7-amino-4-carbamoylmethylcoumarin (ACC).

The PNA identifier tag of the fluorogenic enzyme substrates of the present invention serves two purposes: first, to encode the synthetic history of the organic moiety of the fluorogenic enzyme substrate, and second, to positionally encode the identity of the organic moiety of the fluorogenic enzyme substrate by its location upon hybridization to an oligonucleotide array. The PNA identifier tag is preferably from about 3 to about 50 nucleotides in length, more preferably from about 6 to about 20 nucleotides in length, and even more preferably from about 12 to about 14 nucleotides in length.

In the fluorogenic enzyme substrates, the organic moiety is covalently attached to the fluorogenic moiety and comprises a cleavage recognition site for an enzyme. In a preferred embodiment, the organic moiety comprises a cleavage recognition site for a nucleophilic enzyme. In a more preferred embodiment, the organic moiety comprises a cleavage recognition site for a hydrolase. Suitable hydrolases include, but are not limited to, proteases or (interchangeably) proteinases, peptidases, lipases, nucleases, oligosaccharidases, polysaccharidases, phosphatases, sulfatases, neuraminidases and esterases. In a preferred embodiment, the organic moiety comprises a cleavage recognition site for a protease. Suitable proteases include, but are not limited to, aspartic proteases, cysteine proteases, metalloproteases, threonine proteases and serine proteases.

As such, suitable organic moieties include, but are not limited to, an amino acid, a polypeptide sequence, a nucleotide sequence, a lipid, a carbohydrate and a small organic molecule. In preferred embodiments, the organic moiety is an amino acid, a polypeptide sequence or a small organic molecule having an amide bond that is recognized by a protease. Typically, when the organic moiety is a polypeptide, the polypeptide sequence is covalently attached to the fluorogenic moiety through an amide bond, wherein the amide bond is formed between a carboxylic acid moiety of the carboxy terminus of the polypeptide sequence and an amine of the fluorogenic moiety.

It will be readily apparent to those of skill in the art that depending on the fluorogenic moiety used, the fluorogenic enzyme substrates can comprise more than one organic moiety. For instance, if the fluorogenic moiety is coumarin, the fluorogenic enzyme substrate will comprise one organic moiety. However, if the fluorogenic moiety is, for example, rhodamine, the fluorogenic enzyme substrate will typically comprise two organic moieties. When more than one organic moiety is present, the organic moieties can be the same or different, although in preferred embodiments, the organic moieties are the same.

In one preferred embodiment, the fluorogenic enzyme substrate has the following structure: 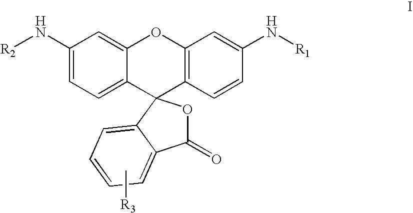
wherein: R¹ and R² are organic moieties including, but not limited to, the following: an amino acid, a polypeptide sequence, a nucleotide sequence, a lipid, a carbohydrate and a small organic molecule; and R³ is a PNA identifier tag. In a preferred embodiment of the fluorogenic enzyme substrate of Formula I, R¹ and R² are both polypeptide sequences, the polypeptide sequences having the following structure:
—C(O)-AA¹-AA²-(AAⁱ)_{J-2}
wherein: each of A¹ through AAⁱ is an amino acid residue which is a member independently selected from the group of natural amino acid residues, unnatural amino acid residues and modified amino acid residues; J denotes the number of amino acid residues forming the polypeptide sequence and is a member selected from the group consisting of the numbers from 2 to 10, such that J-2 is the number of amino acid residues in the polypeptide sequence exclusive of AA¹-AA²; and i denotes the position of the amino acid residue relevant to AA¹ and when J is greater than 2, i is a member selected from the group consisting of the numbers from 3 to 10.

In the above compound of Formula I, cleavage of the amide bond between the rhodamine and the organic moiety (e.g., amino acid, polypeptide or small molecule) by a protease or other such enzyme relieves the suppression of absorbance and fluorescence signal. These properties combined with the favorable spectral properties of rhodamine for use with imaging instruments that utilize the argon-ion laser (488 nm), the stability of rhodamine fluorescence over pH ranges used for most proteases (pH 3 to pH 9), and the red-shifted emission and excitation spectrum of rhodamine that allows for reduced background fluorescence makes the fluorogenic enzyme substrates of Formula I ideal tools for monitoring, for example, protease activity.

In another embodiment, the fluorogenic moiety is a fluorescence donor moiety and the fluorogenic enzyme substrate further comprises a fluorescence acceptor moiety. In this embodiment, the fluorescence acceptor moiety is covalently attached to the fluorescence donor moiety through the organic moiety. In this embodiment, the fluorogenic enzyme substrates of the present invention comprise, in essence, the following: a fluorescence donor moiety; a fluorescence acceptor moiety; an organic moiety comprising a cleavage recognition site for an enzyme, wherein the fluorescence donor moiety is covalently attached to the fluorescence acceptor moiety through the organic moiety; and a petido nucleic acid (PNA) identifier tag covalently attached to the fluorescence donor moiety, wherein the PNA identifier tag identifies the organic moiety.

In another aspect, the present invention provides a method for assaying for the presence of an enzymatically active enzyme in a sample, the method comprising: (a) contacting the sample with a fluorogenic enzyme substrate of the present invention under conditions such that if the enzymatically active enzyme is present in the sample, at least a portion of the organic moiety is cleaved from the fluorogenic moiety of the fluorogenic enzyme substrate, thereby producing a fluorescent compound having the PNA identifier tag covalently attached thereto; (b) hybridizing the fluorescent compound to an array of oligonucleotides; and (c) detecting the fluorescent compound that hybridizes to the array of oligonucleotides, wherein detection of the fluorescent compound indicates the presence of the enzymatically active enzyme in the sample. In one embodiment, the method further comprises: (d) quantifying the fluorescent compound, thereby quantifying the amount of enzymatically active enzyme present in the sample.

The methods of the present invention can be used to assay for any known or later discovered nucleophilic enzymes (e.g., hydrolases, such as proteases, etc.). In one embodiment, the enzymatically active enzyme is a hyrolase, such as a protease. Suitable proteases include, but are not limited to, aspartic proteases, cysteine proteases, metalloproteases, threonine proteases and serine proteases. In one embodiment, the protease is a protease of a microorganism such as bacteria, fungi, yeast, viruses and protozoa.

Suitable samples include, but are not limited, to biological samples such as sputum, blood, blood cells (e.g., white cells), tissue or fine needle biopsy samples, urine peritoneal fluid, pleural fluid or cells therefrom. Biological samples may also include sections of tissue such as frozen sections taken for histological purposes. Similarly, biological samples preferably include cells, tissues and organ lysates.

In still another aspect, the present invention provides a method for detecting activation of a biological pathway by assaying for the presence of an enzymatically active enzyme in a sample, the method comprising: (a) contacting the sample with a fluorogenic enzyme substrate of the present invention under conditions such that if the enzymatically active protease is present in the sample, at least a portion of the organic moiety is cleaved from the fluorogenic moiety of the fluorogenic enzyme substrate, thereby producing a fluorescent compound having the PNA identifier tag covalently attached thereto; (b) hybridizing the fluorescent compound to an array of oligonucleotides; and (c) detecting the fluorescent compound that hybridizes to the array of oligonucleotides, wherein detection of the fluorescent compound indicates the presence of the enzymatically active protease in the sample, and wherein the presence of the enzymatically active protease in the sample indicates activation of the biological pathway.

In yet another embodiment, the present invention provides libraries, arrays or microarrays of the fluorogenic enzyme substrates of the present invention. In one embodiment, the library of fluorogenic enzyme substrates comprises at least a first fluorogenic enzyme substrate and a second fluorogenic enzyme substrate, wherein the first and second fluorogenic enzyme substrates comprise: (a) a fluorogenic moiety; (b) an organic moiety covalently attached to the fluorogenic moiety, wherein the organic moiety comprises a cleavage recognition site for an enzyme; and (c) a petido nucleic acid (PNA) identifier tag covalently attached to the fluorogenic moiety, wherein the PNA identifier tag identifies the organic moiety. Typically, the members of a library will differ from one another in terms of their organic moieties, although they can differ from one another in other respects as well (e.g., they can differ in terms of the fluorogenic moieties). In a preferred embodiment, the organic moieties are polypeptide sequences and the members of the library differ from one another in that each member of the library has a different polypeptide sequence. The differences can reside in the polypeptide sequence, polypeptide length or both.

In a preferred embodiment, the library comprises at least 10 fluorogenic enzyme substrates, more preferably at least 100 fluorogenic enzyme substrates, more preferably at least 103 fluorogenic enzyme substrates, even more preferably at least 104 fluorogenic enzyme substrates, still more preferably 10⁵ fluorogenic enzyme substrates, and even more preferably at least 10⁶ fluorogenic enzyme substrates.

In one embodiment, the present invention provides a library of fluorogenic polypeptides comprising at least a first fluorogenic polypeptide and a second fluorogenic polypeptide, wherein the first and second fluorogenic polypeptides have the following structure: 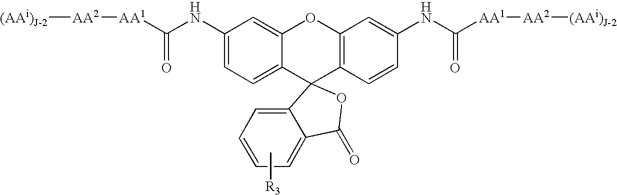
wherein: each AA¹-AA²-(AAⁱ)_{J-2} is a polypeptide sequence, wherein each of AA¹ through AAⁱ is an amino acid residue which is a member independently selected from the group of natural amino acid residues, unnatural amino acid residues and modified amino acid residues; J denotes the number of amino acid residues forming the polypeptide sequence and is a member selected from the group consisting of the numbers from 2 to 10, such that J-2 is the number of amino acid residues in the polypeptide sequence exclusive of AA¹-AA²; and i denotes the position of the amino acid residue relevant to AA¹ and when J is greater than 2, i is a member selected from the group consisting of the numbers from 3 to 10.

In still another aspect, the present invention provides a method for determining a polypeptide sequence specificity profile of an enzymatically active protease, the method comprising: (a) contacting the protease with a library of fluorogenic polypeptides of the present invention, wherein the polypeptide sequences are selectively cleaved by the protease, thereby producing a fluorescent compound having the PNA identifier tag covalently attached thereto; (b) hybridizing the fluorescent compound to an array of oligonucleotides; (c) detecting the fluorescent compound that hybridizes to the array of oligonucleotides; and (d) determining the sequence of the polypeptide sequences, thereby identifying the polypeptide sequence specificity profile of the protease. In one embodiment, this method further comprises: (e) quantifying the fluorescent compound, thereby quantifying the protease.

Other features, objects and advantages of the invention and its preferred embodiments will become apparent from the detailed description, examples, claims and figures that follow.

### BRIEF DESCRIPTION OF THE DRAWINGS

FIG. 1A. The fluorogenic rhodamine peptidyl substrate and its fluorescent counterpart.

FIG. 1B. General structure of a PNA encoded tetrapetidic protease substrate library. The PNA codons P₁-P₄ encode for the peptide sidechains P₁-P₄. The length X of each codon can be adjusted in order to reflect the different importances of the peptide positions P₁-P4 during the hybridization process.

FIG. 1C. Proteolytic cleavage of the PNA encoded substrate library in solution followed by spatial deconvolution on chip.

FIG. 1D. Rhodamine peptidyl protease substrates and their PNA encoded counterparts.

FIG. 2A. Synthesis scheme on solid support of PNA encoded Rhodamine protease substrate library. The Rhodamine scaffold is coupled to the resin, its amino functions are deprotected and the first amino acid is added. After Mtt deprotection the first codon is added. The use of alloc and Fmoc as the orthogonal protection groups PG1 and PG2, respectively, allows for alternating between synthesis of the peptide chain and the PNA codons which is the foundation of a combinatorial split and combine synthesis scheme.

FIG. 2B. Summary of the codons utilized in the synthesis of the 192 member split and combine library. The sequences are listed in the C-terminus to N-terminus direction.

FIG. 3A. Biological activity of the Rhodamine peptidyl protease substrates. The substrates exhibit good selectivity. Only the correct substrate/enzyme pair-1 with caspase-3 and 3 with thrombin—gives rise to a strong fluorescent signal.

FIG. 3B. Comparison of Km, kcat and kcst/Km of Rhodamine protease substrates with and without PNA tag.

FIG. 4A. Thrombin and caspase-3 features on Affymetrix chips loaded with the PNA encoded probes 2 and 4. Only the caspase-3 feature on the cip loaded with sample containing caspase-3 is lightening up in a concentration dependent manner.

FIG. 4B. The caspase-3 feature on the chip loaded with sample containing caspase-3 shows a linear signal increase. The thrombin feature shows a slight increase which is comparable to the background signals without enzyme.

FIG. 5A. The probes 2 and 4 were added to apoptotic and nonapoptotic cell lysates. An increase in fluorescence was monitored in the apoptotic cell lysate indicating the presence of an active protease.

FIGS. 5B and 5C. Spatial deconvolution of the samples on self-printed arrays shows the apoptotic activation of caspase-3. The caspase-3 spots are marked with C[2] and the thrombin spots are marked with T[2].

FIG. 6A. Spatial deconvolution of the 192 member PNA encoded substrate library after incubation with nonapoptotic cell lysate, apoptotic cell lysate, purified caspase-3 or three different proteases with broad specificity resulting in complete hydrolysis of the library.

FIG. 6B. The intensity values derived for purified caspase-3.

FIG. 6C. The difference of the intensity values between nonapoptotic and apoptotic cell lysate for each subarray. The activation of caspase-3 during apoptosis can easily be monitored by comparison of the signals on the PI =aspartic acid subarray of the purified enzyme with the differential signal from the two lysates.

### DETAILED DESCRIPTION OF THE INVENTION AND PREFERRED EMBODIMENTS

### A. DEFINITIONS

All technical and scientific terms used herein generally have the same meaning as commonly understood by one of ordinary skill in the art to which this invention belongs. The present definitions and abbreviations are generally offered to supplement the art-recognized meanings. Generally, the nomenclature used herein and the laboratory procedures organic chemistry, polypeptide synthesis and enzyme chemistry described below are those well known and commonly employed in the art. Generally, enzymatic reactions and purification steps are performed according to the manufacturer's specifications. Standard techniques, or modifications thereof, are used for chemical syntheses and chemical analyses.

The term “monomer(s)” as used relative to organic moiety synthesis or PNA identifier tag synthesis refers to discreet building blocks employed to prepare the organic moiety or the PNA identifier tag of the fluorogenic enzyme substrates of the present invention. Thus, in the case where the organic moiety is a polypeptide, the monomer is typically an amino acid, but can comprise a di- or higher amino acid fragment of the polypeptide that is incorporated into the fluorogenic enzyme substrate as a single entity. In the case of PNA identifier tag synthesis, the monomer is a nucleotide or a string of nucleotides. The term “monomer(s)” is used interchangeably with the term “building block(s),” and both terms are used in connection with the synthesis of the organic moiety comprising the cleavage recognition site for an enzyme as well as the synthesis of the peptido nucleic acid (PNA) identifier tag.

The term “peptido nucleic acid identifier tag” or “PNA identifier tag” or “PNA tag” refer to a PNA sequence that serves two purposes: first, to encode the synthetic history of the organic moiety of the fluorogenic enzyme substrate, and second, to positionally encode the identity of the organic moiety of the fluorogenic enzyme substrate by its location upon hybridization to an oligonucleotide array. As such, in one embodiment, the PNA sequence identifies which monomer reaction a given solid support has experienced in the synthesis of the organic moiety as well as the step in the synthesis series in which the solid support visited the monomer reaction. The PNA identifier tag can be covalently attached to the solid support or, preferably, it can be covalently attached to the fluorogenic moiety of the fluorogenic enzyme substrate of the present invention, through a linker group. A “monomer” of a PNA tag can include a unit of one or more PNAs that identify a particular building block used for compound synthesis. For example, a PNA monomer having a 3-base sequence “ACT” could signify an addition of a lysine residue to an organic moiety.

“Polypeptide” or “peptide” refers to a polymer in which the monomers are amino acids and are joined together through amide bonds, alternatively referred to as a “polypeptide.” When the amino acids are α-amino acids, either the L-optical isomer or the D-optical isomer can be used. Additionally, unnatural amino acids, for example, β-alanine, phenylglycine and homoarginine are also included. Commonly encountered amino acids that are not gene-encoded may also be used in the present invention. All of the amino acids used in the present invention may be either the D- or L-isomer. The L-isomers are generally preferred. In addition, other peptidomimetics are also useful in the present invention. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983).

“Oligonucleotides” refers to a single-stranded DNA or RNA molecule, typically prepared by synthetic means. The oligonucleotides employed in the methods of the present invention will usually be 8 to 150 nucleotides in length, preferably from 10 to 50 nucleotides and more preferably from 12 to 20 nucleotides, although oligonucleotides of different length may be appropriate in some circumstances. Suitable oligonucleotides may be prepared by the phosphoramidite method described by Beaucage et al., Tetr. Lett., 22:1859-1862 (1981), or by the triester method according to Matteucci et al., T. Am. Chem. Soc., 103:3185 (1981), both incorporated herein by reference, or by other methods such as by using commercial automated oligonucleotide synthesizers.

As used herein, the term “linking group” refers to a group that links a fluorogenic enzyme substrate of the present invention to a solid support, a PNA identifier tag to either a solid support or a fluorogenic moiety of the fluorogenic enzyme substrates of the present invention or an organic moiety to a fluorogenic moiety. Linking groups of diverse structures are useful in practicing the present invention. Exemplary linking groups include, but are not limited to, organic functional groups (e.g., —C(O)—, —NR—, —C(O)S—, —C(O)NR—, etc.); substituted or unsubstituted alkyl, substituted or unsubstituted heteroalkyl and substituted or unsubstituted aryl groups each of which are, in addition to other optional substituents, homo- or hetero-disubstituted with organic functional groups, that adjoin the linker arm to, for example, the target compound and the solid support. The linking groups of the invention can include a group that is cleaved by, for example, light, heat, reduction, oxidation, hydrolysis or enzymatic action (e.g., nitrophenyl, disulfide, ester, etc.). Alternatively, the linking group can be substantially stable under a range of conditions. By providing for the use of linkers with a wide range of physicochemical characteristics, selected properties of the compounds of the present invention and their PNA identifier tags can be manipulated. Properties that are amenable to manipulation include, for example, hydrophobicity, hydrophilicity, surface-activity and the distance from the solid support of the species bound to the solid support via the linking group.

The term “substrate” or “solid support” refers to a material having a rigid or semi-rigid surface which contains or can be derivatized to contain reactive fuinctionality that covalently links a fluorogenic enzyme substrate of the present invention or a PNA identifier tag to the surface thereof. Such materials are well known in the art and include, by way of example, silicon dioxide supports containing reactive Si—OH groups, polyacrylamide supports, polystyrene supports, polyethyleneglycol supports, and the like. Such supports will preferably take the form of small beads, pellets, disks, or other conventional forms, although other forms may be used. In some embodiments, at least one surface of the substrate will be substantially flat. In preferred embodiments, the substrate or solid support is roughly spherical.

The term “reactions” refers to any reaction that adds a monomer to the solid support, that modifies the chemical entity formed after monomer addition to the solid support and/or that removes a group from the solid support. The reactions can employ monomers (building blocks) that become incorporated onto the solid support or can merely employ a reagent, such as heat, base, acid, an oxidizing agent, a reducing agent, an enzyme, etc. that does not become incorporated into the structures found on the support. Modifications of the chemical entity formed after monomer addition to the solid support include, for example, cyclization, isomerization, etc. Removal of a group from the solid support includes hydrolysis to remove an ester, removal of protecting groups, etc.

The term “protecting group” or ” compatible protecting group” refers to a chemical group that exhibits the following characteristics: 1) reacts selectively with the desired functionality in good yield to give a derivative that is stable to the projected reactions for which protection is desired; 2) can be selectively removed chemically and/or enzymatically from the derivatized solid support to yield the desired functionality; and 3) is removable in good yield by reagents compatible with the other functional group(s) generated in such projected reactions. Examples of protecting groups can be found in Greene, et al. (1991) Protective Groups in Organic Synthesis, 2nd Ed. (John Wiley & Sons, Inc., New York). Preferred protecting groups include, but are not limited to, acid-labile protecting groups (such as Boc or DMT); base-labile protecting groups (such as Fmoc, Fm, phosphonioethoxycarbonyl (Peoc), etc.); groups which may be removed under neutral conditions (e.g., metal ion-assisted hydrolysis ), such as DBMB, allyl or alloc, 2-haloethyl; groups which may be removed using fluoride ion, such as 2-(trimethylsilyl)ethoxymethyl (SEM), 2-(trimethylsilyl)-ethyloxycarbonyl (Teoc) or 2-(trimethylsilyl)ethyl (Te) S; and groups which may be removed under mild reducing conditions (e.g., with sodium borohydride or hydrazine), such as Lev. Particularly preferred protecting groups include, but are not limited to, Fmoc, Fm, Menpoc, Nvoc, Nv, Boc, CBZ, allyl, alloc (allyloxycarbonyl), Npeoc (4-nitrophenethyloxycarbonyl), Npeom (4-nitrophenethyloxymethyloxy), α,α-dimethyl-3,5-dimethoxybenzyloxycarbonyl (ddz) and trityl groups. The particular removable protecting group employed is not critical to the methods of the present invention.

The term “orthogonal protecting groups” refer to two or more compatible protecting groups which, in the presence of one other, can be differentially removed or, if not differentially removed, can be differentially reprotected. In one embodiment, it may be desirable to remove all of the protecting groups in one step, such as at completion of the synthesis.

“Analyte,” as used herein means any compound or molecule of interest for which a screening assay is performed. In a presently preferred embodiment, the analyte is an enzyme, preferably a nucleophilic enzyme and more preferably a hydrolytic enzyme.

As used herein, “energy transfer” refers to the process by which the fluorescence emission of a fluorescent group is altered by a fluorescence-modifying group. If the fluorescence-modifying group is a quenching group, then the fluorescence emission from the fluorescent group is attenuated (quenched). Energy transfer can occur through fluorescence resonance energy transfer, or through direct energy transfer. The exact energy transfer mechanisms in these two cases are different. It is to be understood that any reference to energy transfer in the instant application encompasses all of these mechanistically-distinct phenomena.

As used herein, “energy transfer pair” refers to any two molecules that participate in energy transfer. Typically, one of the molecules acts as a fluorescent group, and the other acts as a fluorescence-modifying group. There is no limitation on the identity of the individual members of the energy transfer pair in this application. All that is required is that the spectroscopic properties of the energy transfer pair as a whole change in some measurable way if the distance between the individual members is altered by an appropriate amount.

As used herein, “fluorescence-modifying group” refers to a molecule that can alter in any way the fluorescence emission from a fluorescent group. A fluorescence-modifying group generally accomplishes this through an energy transfer mechanism. Depending on the identity of the fluorescence-modifying group, the fluorescence emission can undergo a number of alterations, including, but not limited to, attenuation, complete quenching, enhancement, a shift in wavelength, a shift in polarity, and a change in fluorescence lifetime. One example of a fluorescence-modifying group is a quenching group.

As used herein, “quenching group” or “quenching agent” or “quencher” refers to any fluorescence-modifying group that can attenuate at least partly the light emitted by a fluorescent group. This attenuation is referred to herein as “quenching.” Hence, illumination of the fluorescent group in the presence of the quenching group leads to an emission signal that is less intense than expected, or even completely absent. Quenching typically occurs through energy transfer between the fluorescent group and the quenching group.

“Fluorescence resonance energy transfer” or ‘FRET” refers to an energy transfer phenomenon in which the light emitted by the excited fluorescent group is absorbed at least partially by a fluorescence-modifying group of the invention. If the fluorescence-modifying group is a quenching group, then that group will preferably not radiate a substantial fraction of the absorbed light as light of a different wavelength, and will preferably dissipate it as heat. FRET depends on an overlap between the emission spectrum of the fluorescent group and the absorption spectrum of the quenching group. FRET also depends on the distance between the quenching group and the fluorescent group.

“Moiety” refers to the radical of a molecule that is attached to another moiety. For instance, in the fluorogenic enzyme substrates of the present invention, an organic moiety (e.g., a polypeptide) is covalently attached to a fluorogenic moiety (e.g., rhodamine).

The term “chemical library” or “array” refers to an intentionally created collection of differing fluorogenic enzyme substrates of the present invention that can be prepared synthetically and that can be screened for biological activity in a variety of different formats (e.g., libraries of soluble compounds, libraries of compounds tethered to solid supports, etc.). The library comprises at least 2 members, preferably at least 10 members, more preferably at least 10² members and still more preferably at least 10³ members. Particularly preferred libraries comprise at least 10⁴ members, more preferably 10⁵ members and still more preferably at least 10⁶ members.

A “cleavage recognition site of an enzyme” is a substrate site for the enzyme. The cleavage recognition site can be part of a substrate recognition motif for the enzyme. The substrate recognition motif for the enzyme can be any structure or sequence that is recognized by an enzyme and that directs or helps in the enzymatic modification of the substrate by the enzyme.

A “nucleophilic enzyme” is an enzyme having a nucleophile that plays a role in the enzymatic activity (e.g., hydrolytic activity) of the enzyme. For instance, for serine proteases, such as trypsin, the gamma-oxygen of serine 195 is the nucleophile that catalyzes amide hydrolysis. In a preferred embodiment, the nucleophilic enzyme is a hydrolase, i.e., a hydrolytic enzyme. Examples of hydrolases include, but are not limited to, proteases or (interchangeably) proteinases, peptidases, lipases, nucleases, oligosaccharidases, polysaccharidases, phosphatases, sulfatases, neuraminidases and esterases.

### B. FLUOROGENIC ENZYME SUBSTRATES AND METHOCS OF PREPARATION

In one embodiment, the present invention provides fluorogenic enzyme substrates comprising: (a) a fluogenic moiety; (b) an organic moiety covalently attached to the fluorogenic moiety, wherein the organic moiety comprises a cleavage recognition site for an enzyme; and (c) a petido nucleic acid (PNA) identifier tag covalently attached to the fluorgenic moiety, wherein the PNA identifier tag identifies the organic moiety.

In another embodiment, the present invention provides fluorogenic enzyme substrates comprising: (a) a fluorescence donor moiety; (b) a fluorescence acceptor moiety, wherein the fluorescence acceptor moiety is covalently attached to the fluorescence donor moiety through an organic moiety comprising a cleavage recognition site for an enzyme; and (c) a petido nucleic acid (PNA) identifier tag covalently attached to the fluorgenic moiety, wherein the PNA identifier tag identifies the organic moiety.

The fluorogenic moiety can be any fluorescent substance that emits light at a certain wavelength (emission wavelength) when it is illuminated by light of a different wavelength (excitation wavelength), but that can exist in at least two different states having two different fluorescent properties. For instance, in one embodiment, suitable fluorogenic moieties include those that can exist in a quenched state when they are covalently attached to an organic moiety and a fluorescent state when the organic moiety or a portion thereof is cleaved therefrom by, for example, a hydrolase such as a protease. Similarly, suitable fluorogenic moieties include those that can exist in a quenched state when they are covalently attached to an organic moiety that further comprises a quenching agent, and a fluorescent state when the organic moiety or a portion thereof, together with the quenching agent, is cleaved from the fluorogenic moiety by, for example, a hydrolase such as a protease.

As such, in one embodiment, when the organic moiety is covalently attached to the fluorogenic moiety, the fluorescence signal of the fluorogenic moiety is attenuated, quenched or suppressed. In another embodiment, the organic moiety further comprises a quenching agent, i.e., a quencher, that is capable of quenching the fluorescence of the fluorogenic moiety when the organic moiety is covalently attached to the fluorgenic moiety. Here, the fluorogenic enzyme substrates comprises a fluorescence donor moiety and a fluorescence acceptor moiety.

Many fluorescent moieties suitable for use in the compounds of the present invention are commercially available from the SIGMA chemical company (Saint Louis, Mo.), Molecular Probes (Eugene, Org.), R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersburg, Md.), Fluka Chemica-Biochemika Analytika (Fluka Chemie AG, Buchs, Switzerland), and Applied Biosystems (Foster City, Calif.), as well as many other commercial sources known to one of skill.

Examples of fluorogenic moieties suitable for use in the fluorogenic enzyme substrates of the present invention include, but are not limited to, those set forth in Table I.

```
TABLE I
4-acetamido-4′-isothiocyanatostilbene-2,2′disulfonic acid
acridine and derivatives:
    acridine
    acridine isothiocyanate
5-(2′-aminoethyl)aminonaphthalene-1-sulfonic acid (EDANS)
4-amino-N-[3-vinylsulfonyl)phenyl]naphthalimide-3,5 disulfonate
N-(4-anilino-1-naphthyl)maleimide
anthranilamide
BODIPY
Brilliant Yellow
coumarin and derivatives:
coumarin
    7-amino-4-methylcoumarin (AMC, Coumarin 120)
    7-amino-4-trifluoromethylcouluarin (Coumaran 151)
cyanine dyes
cyanosine
4′,6-diaminidino-2-phenylindole (DAPI)
5′,5″-dibromopyrogallol-sulfonaphthalein (Bromopyrogallol Red)
7-diethylamino-3-(4′-isothiocyanatophenyl)-4-methylcoumarin
diethylenetriamine pentaacetate
4,4′-diisothiocyanatodihydro-stilbene-2,2′-disulfonic acid
4,4′-diisothiocyanatostilbene-2,2′-disulfonic acid
5-[dimethylamino]naphthalene-1-sulfonyl chloride (DNS, dansylchloride)
4-(4′-dimethylaminophenylazo)benzoic acid (DABCYL)
4-dimethylaminophenylazophenyl-4′-isothiocyanate (DABITC)
eosin and derivatives:
    eosin
    eosin isothiocyanate
erythrosin and derivatives:
    erythrosin B
    erythrosin isothiocyanate
ethidium
fluorescein and derivatives:
    5-carboxyfluorescein (FAM)
    5-(4,6-dichlorotriazin-2-yl)aminofluorescein (DTAF)
    2′,7′-dimethoxy-4′5′-dichloro-6-carboxyfluorescein (JOE)
    fluorescein
    fluorescein isothiocyanate
    QFITC (XRITC)
fluorescamine
IR144
IR1446
Malachite Green isothiocyanate
4-methylumbelliferone
ortho cresolphthalein
nitrotyrosine
pararosaniline
Phenol Red
B-phycoerythrin
o-phthaldialdehyde
pyrene and derivatives:
    pyrene
    pyrene butyrate
    succinimidyl 1-pyrene butyrate
quantum dots
Reactive Red 4 (Cibacron ™ Brilliant Red 3B-A)
rhodamine and derivatives:
    6-carboxy-X-rhodamine (ROX)
    6-carboxyrhodamine (R6G)
    lissamine rhodamine B sulfonyl chloride rhodamine (Rhod)
    rhodamine B
    rhodamine 123
    rhodamine X isothiocyanate
    sulforhodamine B
    sulforhodamine 101
sulfonyl chloride derivative of sulforhodamine 101 (Texas Red)
N,N,N′,N′-tetramethyl-6-carboxyrhodamine (TAMRA)
tetramethyl rhodamine
    tetramethyl rhodamine isothiocyanate (TRITC)
riboflavin
rosolic acid
        terbium chelate derivatives
```

In the embodiment of the present invention wherein the organic moiety further comprises a quenching agent, i.e., a quencher, that is capable of quenching the fluorescence of the fluorogenic moiety, the fluorogenic moiety and the quenching agent together comprise the donor-acceptor pair of a fluorescence resonance energy transfer (FRET) pair. FRET is a distance-dependent interaction between the electronic excited states of two dye molecules in which excitation is transferred from a donor molecule to an acceptor molecule without emission of a photon. In general, the primary conditions for FRET are (i) that the donor and acceptor molecules be in close proximity to one another (typically 1 or 10 to 100 or 200 Angstroms); (ii) that the absorption spectrum of the acceptor overlap the fluorescence emission spectrum of the donor; and (iii) that the donor and acceptor transition dipole orientations be approximately or essentially parallel.

There is a great deal of practical guidance available in the literature for selecting appropriate donor-acceptor pairs suitable for use in the fluorogenic enzyme substrates of the present invention, as exemplified by the following references: Pesce et al., Eds., FLUORESCENCE SPECTROSCOPY (Marcel Dekker, New York, 1971); White et al., FLUORESCENCE ANALYSIS: A PRACTICAL APPROACH (Marcel Dekker, New York, 1970); and the like. The literature also includes references providing exhaustive lists of fluorescent and chromogenic molecules and their relevant optical properties for choosing acceptor-donor pairs (see, for example, Berlman, HANDBOOK OF FLUORESCENCE SPECTRA OF AROMATIC MOLECULES, 2nd Edition (Academic Press, New York, 1971); Griffiths, COLOUR AND CONSTITUTION OF ORGANIC MOLECULES (Academic Press, New York, 1976); Bishop, Ed., INDICATORS (Pergamon Press, Oxford, 1972); Haugland, HANDBOOK OF FLUORESCENT PROBES AND RESEARCH CHEMICALS (Molecular Probes, Eugene, 1992) Pringsheim, FLUORESCENCE AND PHOSPHORESCENCE (Interscience Publishers, New York, 1949); and the like.

Generally, it is preferred that an absorbance band of the quencher (i.e., acceptor) substantially overlap the fluorescence emission band of the donor, i.e., the fluorogenic moiety. When the donor (fluorophore) is a component of a fluorogenic enzyme substrate of the present invention that utilizes donor-acceptor energy transfer, the donor fluorescent moiety and the quencher (acceptor) are preferably selected so that the donor and acceptor moieties exhibit donor-acceptor energy transfer when the donor moiety is excited. One factor to be considered in choosing the fluorophore-quencher pair is the efficiency of donor-acceptor energy transfer between them. Preferably, the efficiency of FRET between the donor and acceptor moieties is at least 10%, more preferably at least 50% and even more preferably at least 80%. The efficiency of FRET can easily be empirically tested using methods known in the art.

The efficiency of energy transfer between the donor-acceptor pair can also be adjusted by changing the ability of the donor and acceptor groups to dimerize or closely associate. If the donor and acceptor moieties are known or determined to closely associate, an increase or decrease in association can be promoted by adjusting the length of a linker moiety, or of the organic moiety itself, between the donor and acceptor. The ability of donor-acceptor pair to associate can also be increased or decreased by tuning the hydrophobic or ionic interactions, or the steric repulsions in the probe construct. Thus, intramolecular interactions responsible for the association of the donor-acceptor pair can be enhanced or attenuated. Thus, for example, the association between the donor-acceptor pair can be increased by, for example, utilizing a donor bearing an overall negative charge and an acceptor with an overall positive charge.

Suitable donor and acceptor pairs can be readily selected by those of skill in the art from the list provided in Table I. Examples of suitable donor and acceptor pairs include, but are not limited to, the following: fluorescein and tetramethylrhodamine; 5-(2′-aminoethyl) aminonaphthalene-1-sulfonic acid (EDANS) and fluorescein; EDANS and 4-(4′-dimethylaminopheylazo)benzoic acid (DABCYL); fluorescein and QSY 7 (7 carboxylic acid, succinimidyl ester) dye; and fluorescein and QSY 9 (7 carboxylic acid, succinimidyl ester) dye. Other fluorogenic moieties and quenching agents suitable for use in the present invention are known to those of skill in the art.

In a preferred embodiment, the fluorogenic moiety is one that can exist in a quenched state when it is covalently attached to an organic moiety (e.g., a polypeptide) and a fluorescent state when the organic moiety is cleaved therefrom by, for example, a hydrolase such as a protease. In this embodiment, the fluorescence signal of the fluorogenic moiety is quenched or suppressed when the organic moiety is covalently attached to the fluorogenic moiety and, thus, the use of a quenching dye molecule is not required. Fluorogenic moieties suitable for use in this embodiment of the present invention include, for example, rhodamine dyes and coumarin dyes. In a preferred embodiment, the fluorogenic moiety is a rhodamine moiety, such as rhodamine NHS ester. In another preferred embodiment, the fluorogenic moiety is a coumarin moiety, such as 7-amino-4-methylcoumarin (AMC), 7-amino-4-trifluoromethylcoumarin (AFC), 7-amino-4-chloromethylcoumarin (CMAC) and 7-amino-4-carbamoylmethylcoumarin (ACC). Many suitable forms of these dye compounds are widely commercially available with substitutents on their phenyl moieties that can be used for covalently attaching both the organic moiety and the PNA identifier tag.

As mentioned above, the fluorogenic compounds of the present invention comprise an organic moiety that is covalently attached to the fluorogenic moiety, wherein the organic moiety comprises a cleavage recognition site for an enzyme. Thus, the organic moiety can be any molecule, compound or fragment of a compound that comprises a cleavage recognition site and that can serve as a substrate for an enzyme. The cleavage recognition site can be a portion of the organic moiety or, alternatively, it can be all of the organic moiety. In the former embodiment, the cleavage recognition site can be part of a substrate recognition motif for the enzyme.

In a preferred embodiment, the organic moiety comprises a cleavage recognition site for a nucleophilic enzyme. In a preferred embodiment, the nucleophilic enzyme is a hydrolytic enzyme, i.e., a hydrolase. Examples of hydrolases include, but are not limited to, the following: proteases or (interchangeably) proteinases, peptidases, lipases, nucleases, oligosaccharidases, polysaccharidases, phosphatases, sulfatases, neuraminidases and esterases. As such, depending on the hydrolase being assayed, the organic moiety can be a polypeptide, a lipid, a carbohydrate, an ester, a nucleic acid, a small organic molecule, etc. For instance, if a protease is the hydrolase being assayed, the organic moiety can be a polypeptide or protein. Alternatively, if a protease is the hydrolase being assayed, the organic moiety can be a small organic molecule having an amide bond that is recognized by the protease. If a lipase is the hydrolase being assayed, the organic moiety can be a lipid or a fragment thereof. If cellulase is the hydrolase being detected, the organic moiety can be cellulose. If a lysozyme is the hydrolase being detected, the organic moiety can be bacterial cell wall peptidoglycan. If a phosphatase is the hydrolase being assayed, the organic moiety can be, a compound, such as a small organic molecule, having a phosphate ester.

In a presently preferred embodiment, the hydrolytic enzyme is a protease and the organic moiety is a polypeptide comprising a cleavage recognition site for the protease. Suitable proteases include, but are not limited to, the following: aspartic proteases, cysteine proteases, metalloproteases, threonine proteases and serine proteases. Many protease cleavage sites are known in the art, and these and other cleavage sites can comprise all or a portion of the organic moiety of the fluorogenic enzyme substrates of the present invention. In a preferred embodiment, the present invention provides a library or array of fluorogenic polypeptide substrates, wherein each member of the library has a different polypeptide sequence.

In addition to comprising a fluorogenic moiety and an organic moiety, the compounds of the present invention also comprise a peptido nucleic acid (PNA) identifier tag that identifies the organic moiety and, in preferred embodiments, serves to positionally encode the identity of the organic moiety by its location upon hybridization to an oligonucleotide array. In one embodiment, the PNA identifier tag is covalently attached to the fluorogenic moiety.

The length of the PNA identifier tag can vary. Typically, the PNA identifier tag is from about 3 to about 50 nucleotides in length. In a preferred embodiment, the PNA identifier tag is from about 6 to about 20 nucleotides in length. In another preferred embodiment, the PNA identifier tag is about 12, 13 or 14 nucleotides in length.

Typically, because the cleavage recognition sites, i.e., substrates, for many hydrolases are oligomeric in nature, generation of the compounds of the present invention having different or varying organic moieties, i.e., hydrolytic substrates, is a natural candidate for combinatorial chemistry. For instance, if the hydrolase to be screened for in a given sample is a protease, or if multiple proteases are to screened for in a given sample, then the compounds of the present invention having varying polypeptide sequences can be readily generated using combinatorial chemistry techniques.

Suitable combinatorial chemistry techniques include, for example, the “split-pool” techniques disclosed and claimed in U.S. patent application Ser. No. 10/165,215, the teachings of which are incorporated herein by reference for all purposes, which involve linking a PNA identifier tag to a small molecule or oligomer (e.g., a polypeptide) or, alternatively, to the solid supports that indicate the monomer reactions and corresponding step numbers that define each small molecule or oligomer in the library. For instance, in one embodiment, U.S. patent application Ser. No. 10/165,215 provides a method for preparing a library of diverse compounds, each of the compounds being produced by the step-by-step assembly of building blocks, the method comprising the steps of: (a) apportioning solid supports among a plurality of reaction vessels; and (b) in each reaction vessel of the plurality of reaction vessels, exposing the solid supports to a first building block of a compound and to a first monomer of a peptido nucleic acid (PNA) identifier tag under conditions suitable for immobilization of the first building block and the first monomer, wherein the first building block present in one reaction vessel is different from the first building block present in at least one of the other reaction vessels, wherein the first building block of the compound is capable of being covalently coupled to a second building block and wherein the first monomer of the PNA identifier tag is capable of being covalently coupled to a second monomer. In one embodiment, the method further comprises: (c) pooling the solid supports. In another embodiment, the method further comprises: (c) cleaving the first compound from the solid support. In some embodiments, the methods further comprise: (d) reapportioning the pooled solid supports among a plurality of reaction vessels; and, (e) in each reaction vessel of the plurality of reaction vessels, exposing the solid supports to at least a second building block of the compound and to at least a second monomer of the PNA identifier tag under conditions suitable for attachment of the second building block to the first building block of the compound and the second monomer to the first monomer of the PNA identifier tag, wherein the second building block present in one reaction vessel is different from the second building block present in at least one of the other reaction vessels. As will be appreciated by those of skill in the art, the foregoing steps can be repeated until the desired library of compounds has been generated.

Similar “split-pool” or “split and combine” techniques can be used to prepare the fluorogenic enzyme substrates of the present invention as well as libraries of fluorogenic enzyme substrates. Such techniques can be used to synthesis the fluorogenic enzyme substrates and, in particular, to link a PNA identifier tag to, for example, the fluorogenic moiety of the fluorogenic enzyme substrates of the present invention. By tracking the synthesis pathway that each organic moiety has taken using the PNA identifier tag, one can deduce the sequence of monomers of any organic moiety and, in turn, the identity of the organic moiety present in the fluorogenic enzyme substrate of the present inveniton. As explained herein, once the screening assay has been carried out, one “reads” the PNA identifier tag(s) associated with the organic moiety. In a preferred embodiment, the PNA identifier tag(s) is read by hybridizing the fluorogenic enzyme substrates of the present invention to a spatially addressable oligonucleotide array.

The PNA identifier tag can be associated with the fluorogenic moiety through a variety of mechanisms, either directly, through a linking group, or through a solid support upon which the fluorogenic substrates of the present invention is synthesized. In a preferred embodiment, the PNA identifier tag is associated with the fluorogenic moiety such that when the fluorogenic substrate of the present invention is removed from the solid support, the PNA identifier tag is attached to the fluorogenic moiety, typically through a linking group. In this manner, the fluorogenic enzyme substrates can be advantageously used to detect enzyme activity in solution. It is important to note that the PNA identifier tag does not interfere with the biological activity and/or properties of the organic moiety or the enzyme being screened.

A given monomer unit of the PNA tag can be a single PNA base (i.e., a single nucleotide) or a string of PNA bases (ie., a string of nucleotides that are, e.g., 2, 3, 4 or nucleotides in length) that are attached to the fluorogenic moiety or the solid support as a single entity. In a preferred embodiment, a given monomer unit of the PNA tag is a string of PNA bases that are added as a single entity. It will be readily apparent to those of skill that when only a small number of monomer units of an organic moiety or oligomer is varied, one may need to identify only those monomers which vary among the organic moieties or oligomers, as when one wants to vary only a few amino acids in a polypeptide. For instance, one might want to change only 3 to 6 amino acids in a polypeptide that is 6 to 12 amino acids long, or one might want to change as few as 5 amino acids in a polypeptide that is 50 amino acids long. One may uniquely identify the sequence of each polypeptide by providing for each fluorogenic moiety or solid support a PNA identifier tag specifying only the amino acids varied in each sequence, as will be readily appreciated by those skilled in the art. In such cases, all solid supports may remain in the same reaction vessel for the addition of common monomer units and apportioned among different reaction vessels for the addition of distinguishing monomer units.

In view of the foregoing, there are several ways that the PNA can be used as identifier tags. In one embodiment, the PNA can be assembled base-by-base before, during, or after the corresponding organic moiety or oligomer (e.g., polypeptide) synthesis step. In one case of base-by-base synthesis, the tag for each step is a single nucleotide, or at most a few nucleotides (i.e., 2 to 5). This strategy preserves the order of the steps in the linear arrangement of the PNA chain grown in parallel with the organic moiety. In another embodiment, a block-by-block approach is employed. In this embodiment, sets or blocks of PNAs (e.g., 2, 3, 4 or 5 to 10 or more bases) are added as protected, activated blocks. Each block carries the monomer-type information, and the order of addition represents the order of the monomer addition reaction. Alternatively, the block may encode the oligomer synthesis step number as well as the monomer-type information.

As noted above, in preferred embodiment, the PNA identifier tags are attached to chemically reactive groups on the fluorogenic moiety, typically through a linker. Again, in this embodiment, when the fluorogenic enzyme substrate of the present invention is removed from the solid support used to carry out its synthesis, the PNA identifier tag remains attached to the fluorogenic moitey. The size and composition of the library of fluorogenic enzyme substrates of the present invention will be determined by the number of coupling steps and the monomers used during the synthesis.

In addition to encoding the synthetic history of the organic moiety or oligomer, the PNA identifier tag of the present invention also serves to positionally encode the identity of the organic moiety by its location upon hybridization to an oligonucleotide array. The sequences of the PNA identifier tags are initially selected such that they are capable of hybridizing to know sequences on the oligonucleotide array. Methods of making arrays of oligonucleotides are known to those of skill in the art (see, e.g. U.S. Pat. No. 5,143,854, the teachings of which are incorporated herein by reference). Moreover, arrays of oligonucleotides are available from a number of commercial sources, such as Affymetrix (Santa Clara, Calif.). In a preferred embodiment, a GenFlex™ tag array, which is commercially available from Affymetrix, is employed (arrays of this type are currently available at a density of 400,000 features/cm²; the sequences of the chip's probes are available from Affymetrix). In the GenFlex™ tag array, the oligonucleotides are about 20 nucleotides in length and, thus, the sequences of the PNA identifier tag can be selected to hybridize to the full-length sequences of the oligonucleotide probes or to a portion of the sequences of the oligonucleotide probes. In a preferred embodiment, the PNA sequences are selected to hybridize to the terminal 12 residues of the 20 mer probes of a GenFlex™ tag array.

Once the PNA identifier tags have hybridized to the array of oligonucleotides, they can be detected using a variety of different means. Means of detecting fluorescent moieties are well known to those of skill in the art. Thus, for example, fluorescent labels can be detected by exciting the fluorophore with the appropriate wavelength of light and detecting the resulting fluorescence. The fluorescence can be detected visually, by means of photographic film, by the use of electronic detectors such as charge coupled devices (CCDs) or photomultipliers and the like. Other detection systems suitable for use in the methods of the present invention will be readily apparent to those of skill in the art.

In a presently preferred embodiment, the fluorogenic moiety is rhodamine and the fluorogenic enzyme substrate has the following structure: 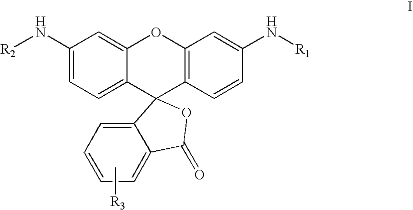
wherein: R¹ and R² are independently selected and include, but are not limited to, an amino acid, a polypeptide sequence, a nucleotide sequence, a lipid, a carbohydrate and a small organic molecule; and R³ is a PNA identifier tag.

In a presently preferred embodiment, the fluorogenic moiety is a rhodamine dye and the organic moiety is a polypeptide having a cleavage recognition site for a protease. In this embodiment, two polypeptide sequences are covalently attached to the rhodamine through amide bonds, wherein the amide bonds are formed between the carboxylic acid moieties of the carboxy terminus of the polypeptide sequences and amines of the rhodamine. In a presently preferred embodiment, the two polypeptide sequences are the same. As such, in a presently preferred embodiment, the compounds of the present invention have the following structure: 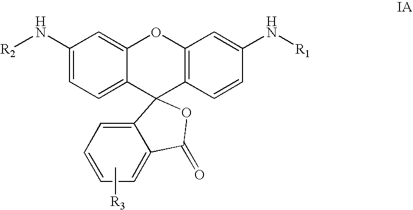
wherein: R¹ and R² are both polypeptide sequences, the polypeptide sequences having the following structure:
C(O)-AA¹-AA²-(AAⁱ)_{J-2}
wherein: AA¹-AA²-(AAⁱ)_{J-2} is a polypeptide sequence, wherein each of AA¹ through AAⁱ is an amino acid residue including, but not limited to, natural amino acid residues, unnatural amino acid residues and modified amino acid residues; J denotes the number of amino acid residues forming the polypeptide sequence and is an integer having a value ranging from about 2 to about 10, such that J-2 is the number of amino acid residues in the polypeptide sequence exclusive of AA¹-AA²; i denotes the position of the amino acid residue relevant to AA¹ and when J is greater than 2, i is a member selected from the group consisting of the numbers from 3 to 10; and R³ is a PNA identifier tag.

In the above compound of Formula IA, cleavage of the amide bond between the rhodamine and the organic moiety (e.g., amino acid, polypeptide, small molecule, etc.) by a protease or other such enzyme relieves the suppression of absorbance and fluorescence signal. These properties combined with the favorable spectral properties of rhodamine for use with imaging instruments that utilize the argon-ion laser (488 nm), the stability of rhodamine fluorescence over pH ranges used for most proteases (pH 3 to pH 9), and the red-shifted emission and excitation spectrum of rhodamine that allows for reduced background fluorescence makes the compounds of Formula IA ideal tools for monitoring protease activity.

In another presently preferred embodiment, the fluorogenic moiety is coumarin and the fluorogenic enzyme substrate has the following structure: 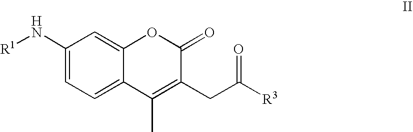
wherein: R¹ is an organic moiety and includes, but is not limited to, an amino acid, a polypeptide sequence, a nucleotide sequence, a lipid, a carbohydrate and a small organic molecule; and R³ is a PNA identifier tag. In a preferred embodiment, R¹ is a polypeptide sequence.

In yet another presently preferred embodiment, the fluorogenic moiety comprises a fluorescence donor moiety and a fluorescence acceptor moiety, wherein the fluorescence donor moiety is rhodamine and the fluorogenic enzyme substrate has the following structure: 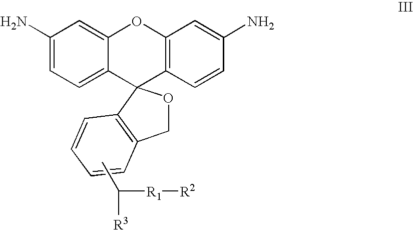
wherein: R¹ is a member selected from the group consisting of an amino acid, a polypeptide sequence, a nucleotide sequence, a lipid, a carbohydrate and a small organic molecule; R² is a fluorescence acceptor moiety; and R³ is a PNA identifier tag. In a preferred embodiment, R¹ is a polypeptide sequence.

In yet another presently preferred embodiment, the fluorogenic moiety comprises a fluorescence donor moiety and a fluorescence acceptor moiety, wherein the fluorescence donor moiety is coumarin and the fluorogenic enzyme substrate has the following structure: 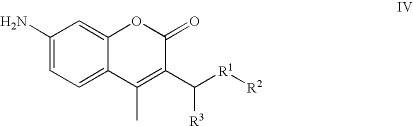
wherein: R¹ is a member selected from the group consisting of an amino acid, a polypeptide sequence, a nucleotide sequence, a lipid, a carbohydrate and a small organic molecule; R² is a fluorescence acceptor moiety; and R³ is a PNA identifier tag. In a preferred embodiment, R¹ is a polypeptide sequence.

As mentioned above, the present invention also provides libraries or arrays of fluorogenic enzyme substrates. In one embodiment, each member of the library has a different organic moiety (e.g., a different polypeptide sequence). As such, in one embodiment, the library of fluorogenic enzyme substrates comprises at least a first fluorogenic enzyme substrate and a second fluorogenic enzyme substrate, wherein the first and second fluorogenic enzyme substrates comprise: (a) a fluorogenic moiety; (b) an organic moiety covalently attached to the fluorogenic moiety, wherein the organic moiety comprises a cleavage recognition site for an enzyme; and (c) a petido nucleic acid (PNA) identifier tag covalently attached to the fluorogenic moiety, wherein the PNA identifier tag identifies the organic moiety. Typically, the members of a library will differ from one another in terms of their organic moieties, although they can differ from one another in other respects as well (e.g., they can differ in terms of the fluorogenic moieties). In a preferred embodiment, the organic moieties are polypeptide sequences and the members of the library differ from one another in that each member of the library has a different polypeptide sequence. The differences can reside in polypeptide sequence, polypeptide length or both.

In a preferred embodiment, the present invention provides a library of fluorogenic polypeptides comprising at least a first fluorogenic polypeptide and a second fluorogenic polypeptide, wherein the first and second fluorogenic polypeptides have the following structure: 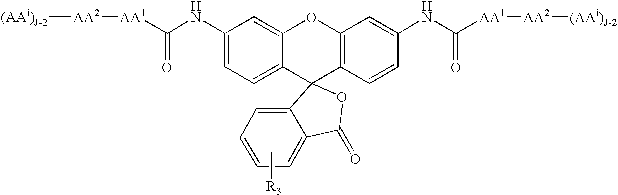
wherein: each AA¹-AA²-(AAⁱ)_{J-2} is a polypeptide sequence, wherein each of AA¹ through AAⁱ is an amino acid residue which is a member independently selected from the group of natural amino acid residues, unnatural amino acid residues and modified amino acid residues; J denotes the number of amino acid residues forming the polypeptide sequence and is a member selected from the group consisting of the numbers from 2 to 10, such that J-2 is the number of amino acid residues in the polypeptide sequence exclusive of AA¹-AA²; and i denotes the position of the amino acid residue relevant to AA¹ and when J is greater than 2, i is a member selected from the group consisting of the numbers from 3 to 10.

In one embodiment, an amino acid residue selected from the group consisting of AA¹, AA², AAⁱ and combinations thereof of the polypeptide sequences of the first polypeptide is a different amino acid residue than an amino acid residue at a corresponding position relative to AA¹ of the polypeptide sequences of the second polypeptide. AA¹ of the polypeptide sequences of the first polypeptide and AA¹ of the polypeptide sequences of the second polypeptide are identical. AA¹ of the polypeptide sequences of the first polypeptide and AA¹ of the polypeptide sequences of the second polypeptide are different.

In other preferred embodiments, the present invention provides other libraries of fluorogenic enzyme substrates having, for example, the structures of the compounds of Formulae II-IV, wherein each of the members of the library has a different organic moiety. Thus, in a preferred embodiment, the library includes at least 10 members, wherein each of the members has a different organic moiety. More preferably, the library includes at least 100 members, more preferably at least 1,000, still more preferably, at least 10,000, more preferably, at least 100,000 and even still more preferably, at least 1,000,000, wherein each of the members of the library has a different. organic moiety (e.g., polypeptide sequence).

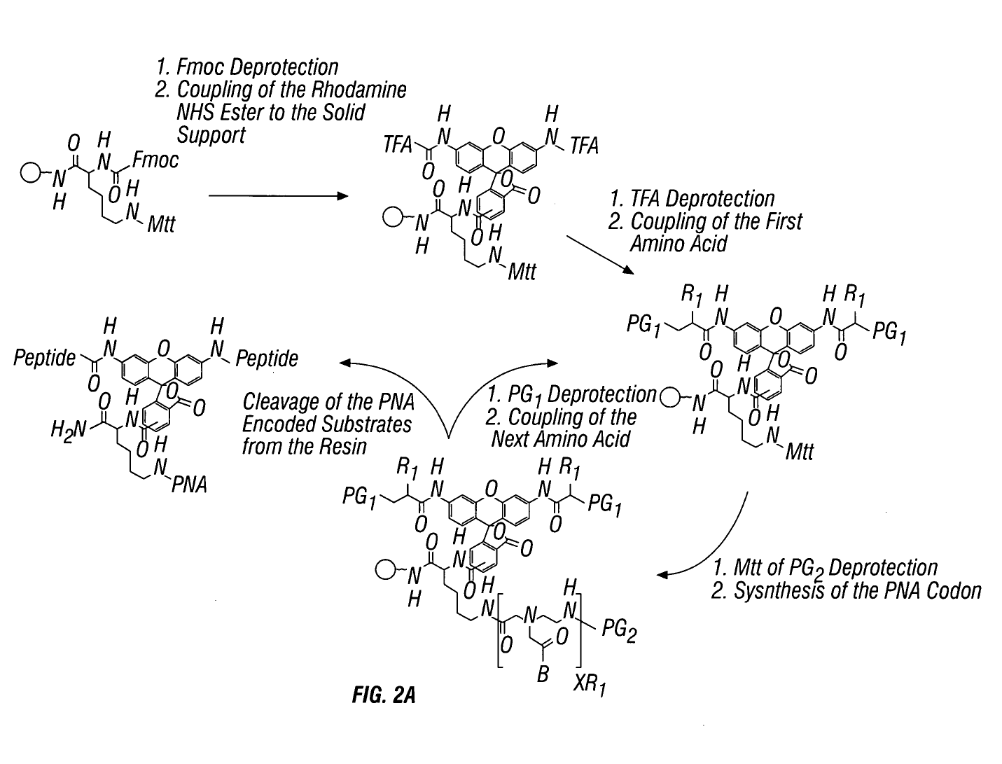

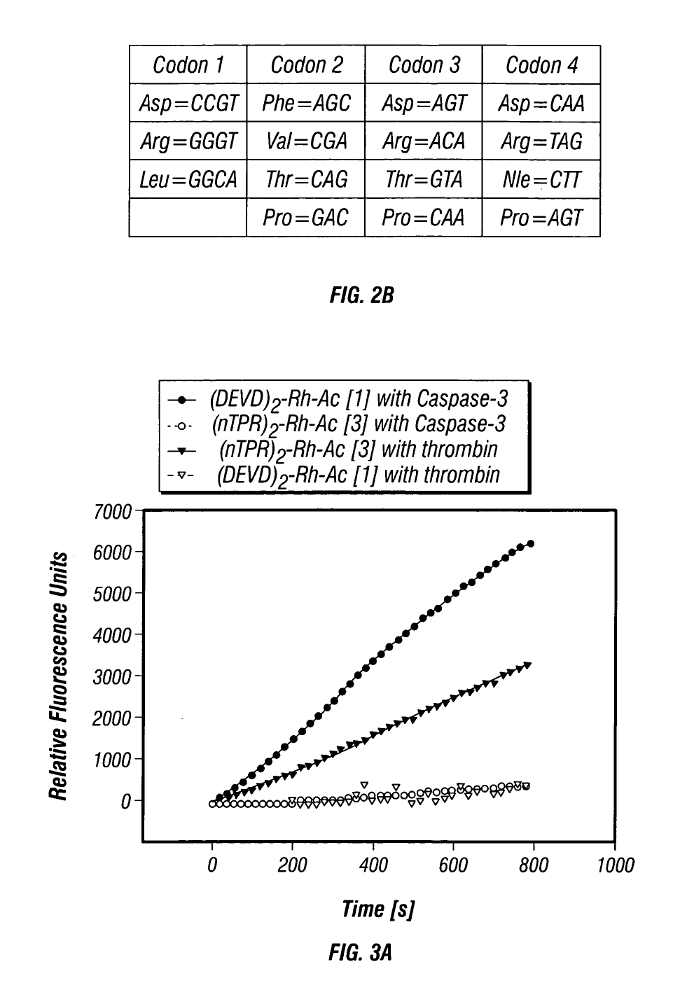

The fluorogenic enzyme substrates of the present invention, as exemplified by the compounds of Formulae I-IV as well as libraries of the compounds of Formulae I-IV, are synthesized by an appropriate combination of generally well-known synthetic methods. Techniques useful in synthesizing the fluorogenic enzyme substrates of the invention are both readily apparent and accessible to those of skill in the relevant art. As mentioned above, the fluorogenic enzyme substrates of the present invention can be synthesized in a combinatorial format using the split-pool techniques disclosed in U.S. patent application Ser. No. 10/165,215, the teachings of which are incorporated by reference. In addition, the fluorogenic enzyme substrates of the present invention, as exemplified by the compounds of Formulae I-IV, can be serially synthesized using the synthesis scheme set forth in FIG. 2. The synthesis scheme of FIG. 2 is offered to illustrate certain of the diverse methods available for use in assembling the fluorogenic enzyme substrates of the present invention, it is not intended to define the scope of reactions or reaction sequences that are useful in preparing the compounds of the present invention. Moreover, other preferred methods for preparing the rhodamine enzyme substrates of the present invention are disclosed in U.S. Provisional Patent Application No. 60/487,331, entitled “METHOD FOR THE PREPARATION OF RHODAMINE,” filed on Jul. 14, 2003 and bearing Attorney Docket No. 021288-003300US, the teachings of which are incorporated herein by reference.

### C. ASSAYS FOR SCREENING FOR BIOLOGICAL ACTIVITIES

The fluorogenic enzyme substrates of the present invention can be used in both in vitro and in vivo enzymatic assays. More particularly, the fluorogenic substrates provided by the present invention can be used to monitor hydrolytic activity (e.g., proteolytic activity) in vitro and in vivo from purified enzymes to complex biological mixtures to whole organisms.

As such, in one embodiment, the present invention provides a method for assaying for the presence of an enzymatically active enzyme in a sample, the method comprising: (a) contacting the sample with a compound comprising (1) a fluorogenic moiety; (2) an organic moiety; and (3) a PNA identifier tag under conditions such that if the enzymatically active enzyme is present in the sample, the organic moiety (or a portion thereof) is cleaved from the fluorogenic moiety of the compound, thereby producing a fluorescent compound having the PNA identifier tag covalently attached thereto; (b) hybridizing the fluorescent compound to an array of oligonucleotides; and (c) detecting the fluorescent compound that hybridizes to the array of oligonucleotides, wherein detection of the fluorescent compound indicates the presence of the enzymatically active enzyme in the sample. In a preferred embodiment, the enzyme is a nucleophilic enzyme. In a more preferred embodiment, the enzyme is a hydrolytic enzyme, i.e., a hydrolase. In a further preferred embodiment, the enzyme is a protease. It will be apparent to those of skill that the methods of the present invention can be used to assay for any known or later discovered nucleophilic enzyme (e.g., hydrolases, such as proteases, etc.).

The term “sample” or, alternatively, “biological sample,” as used herein, refers to a sample obtained from an organism or from components (e.g., cells) of an organism. The sample may be any biological tissue or fluid. Frequently, the sample will be a “clinical sample,” which is a sample derived from a patient. Such samples include, but are not limited to, sputum, blood, blood cells (e.g., white cells), tissue or fine needle biopsy samples, urine peritoneal fluid, pleural fluid or cells therefrom. Biological samples may also include sections of tissue such as frozen sections taken for histological purposes.

In a further preferred embodiment, the method further comprises quantitating the amount of enzyme present in the sample. In a preferred embodiment, the amount of enzyme activity in the sample is determined as a function of the degree of fluorescence in the sample, wherein the amount of fluorescence in the sample is compared with the amount of fluorescence that is present for a standard activity for a known amount of a given enzyme.

Typically, the enzymatic activity (e.g., proteolysis) is measured by the level of fluorescence upon hybridization of the sample to an oligonucleotide microarray. While detection of the fluorescent compound is preferably accomplished using a fluorometer (e.g., a spectrofluorometer), detection may by a variety of other methods well known to those of skill in the art. Thus, for example, since the fluorescent compounds emit in the visible wavelengths, detection may be simply by visual inspection of fluorescence in response to excitation by a light source. Detection may also be by means of an image analysis system utilizing a video camera interfaced to a digitizer or other image acquisition system. Detection may also be by visualization through a filter, as under a fluorescence microscope. The microscope may provide a signal that is simply visualized by the operator. Alternatively, the signal may be recorded on photographic film or using a video analysis system. The signal may also simply be quantified in real-time using either an image analysis system or a photometer.

In another embodiment, the assay methods of the present invention can be used to determine whether an agent modulates, i.e., alters, the activity of an enzyme. For instance, the assay methods can be used to determine whether an agent inhibits an enzyme or, alternatively, whether an agent activates the enzyme. As such, in one embodiment, the present invention provides a method for determining whether an agent modulates the activity of an enzyme, the method comprising: (a) contacting the enzyme and the agent with a fluorogenic enzyme substrate, the fluorogenic enzyme substrate comprising (1) a fluorogenic moiety; (2) an organic moiety covalently attached to the fluorogenic moiety, the organic moiety comprising a cleavage recognition site for an enzyme; and (3) a PNA identifier tag covalently attached to the fluorogenic moiety, the PNA identifier tag identifying the organic moiety, wherein the contacting is under conditions that allow for the organic moiety to be cleaved from the fluorogenic moiety in the presence of the enzyme; (b) hybridizing the compound to an array of oligonucleotides; (c) detecting the presence of fluorescence; and (d) determining whether the agent modulates the activity of the enzyme by comparing the amount of fluorescence in the presence and absence of the agent, wherein a difference between the measured amount of fluorescence in the presence and absence of the agent (i.e., the control) indicates that the agent modulates the activity of the enzyme.

In a preferred embodiment, the amount of enzyme activity in the sample is determined as a function of the degree of fluorescence in the sample, and the amount of enzyme activity in the sample is compared with a standard activity for the same amount of the enzyme. A difference between the amount of enzyme activity in the sample in the presence of the agent and the standard activity in the sample in the absence of the agent indicates that the agent or compound alters the activity of the enzyme.

In another embodiment, the assay methods of the present invention can be used to detect activation of a biological pathway by assaying for the presence of an enzymatically active enzyme in a sample. For example, assaying for the presence of enzymatically active caspase in a sample can be used to detect activation of apoptosis. The methods of the present invention can also be advantageously used to detect activation of other biological pathways, including, for example, hemostasis, blood coagulation, immunological processes, ubiquitination, proteolysis, cell division, cell growth, signaling cascades, the processing of antigens for presentation on the surface of cells, differentiation pathways, survival pathways, neurotransmitter release, cell migration, cell adhesion, complement activation, stress-response pathways, metabolic pathways, and others.

As such, in one embodiment, the present invention provides a method for detecting activation of a biological pathway by assaying for the presence of an enzymatically active enzyme in a sample, the method comprising: (a) contacting the sample with a fluorogenic enzyme substrate, the fluorogenic enzyme substrate comprising (1) a fluorogenic moiety; (2) an organic moiety covalently attached to the fluorogenic moiety, the organic moiety comprising a cleavage recognition site for an enzyme; and (3) a PNA identifier tag covalently attached to the fluorogenic moiety, the PNA identifier tag identifying the organic moiety, wherein the contacting is under conditions that allow for the organic moiety to be cleaved from the fluorogenic moiety in the presence of the enzyme; (b) hybridizing the compound to an array of oligonucleotides; and (c) detecting the fluorescent compound that hybridizes to the array of oligonucleotides, wherein detection of the fluorescent compound indicates the presence of the enzymatically active enzyme (e.g., a protease) in the sample, and wherein the presence of the enzymatically active protease in the sample indicates activation of the biological pathway.

In still another embodiment, the present invention provides a method for determining a polypeptide sequence specificity profile of an enzymatically active protease, the method comprising: (a) contacting the protease with a library of fluorogenic polypeptides of the present invention, wherein the polypeptide sequences are selectively cleaved by the protease, thereby producing a fluorescent compound having the PNA identifier tag covalently attached thereto; (b) hybridizing the fluorescent compound to an array of oligonucleotides; (c) detecting the fluorescent compound that hybridizes to the array of oligonucleotides; and (d) determining the sequence of the polypeptide sequences, thereby identifying the polypeptide sequence specificity profile of the protease. In one embodiment, this method further comprises: (e) quantifying the fluorescent compound, thereby quantifying the protease. Using the foregoing method as well as similar methods using libraries of other fluorogenic enzyme substrates, one can readily probe the reactivity and substrate specificity of enzymes and, in particular, proteases.

The invention will be described in greater detail by way of specific examples. The following examples are offered for illustrative purposes, and are not intended to limit the invention in any manner. Those of skill in the art will readily recognize a variety of non-critical parameters that can be changed or modified to yield essentially the same results.

### D. EXAMPLES

1. Materials and Methods

a) Materials

HATU, Fmoc protected PNA monomers with exocyclic amino functions blocked with the Bhoc group and Fmoc-AEEA-OH PNA spacer and the other reagents for PNA synthesis were obtained from Applied Biosystems (Foster City, Calif.). Rink amide AM resin and Fmoc amino acid derivatives and HOBt were from Novabiochem (San Diego, Calif.). Solvents and reagents for polypeptide synthesis and buffer substances were from Fluka and Aldrich (Milwaukee, Wis.). 3′-Amino-modified 2′-deoxyribo-oligonucleotides were from MWG (High Point, N.C.) or IDT (Coralville, Iowa). Thrombin was from Haematologic Technologies (Essex Jct., Vt.) and caspase-3 was recombinantly expressed and purified by similar methods as those described by Zhou et al. (J. Biol. Chem., 272:7797-7800 (1997)). If not stated otherwise, all reactions were carried under inert atmosphere in an Argonaut Quest 210 Organic Synthesizer.

Abbreviations: Ac: Acetyl; Bhoc: Benzhydryloxycarbonyl; C[2]: Spot for the caspase-3 specific probe 2; CAB: Caspase-3 buffer; CASP: Caspase-3; CHB: Chip hydration buffer; DIEA: Di-isopropylethylamine; DMF: N,N-Dimethylformamide; DMSO: Dimethylsulfoxide; Fmoc: 9-Fluorenylmethoxycarbonyl; HATU: N-[(Dimethylamino)-1H-1,2,3,-trizolo[4,5-b]pyridine-1-ylmethylene]-N-methylmethanaminium Hexafluorophosphonate N-oxide; HEPES: (N-[2-Hydroxyethyl]piperazine-N′-[2-ethanesulfonic acid]); HOBt: N-Hydroxybenzotriazole; MES: (2-[N-Morpholino]ethanesulfonic acid); Mtt: 4-Methyltrityl; n, Nle: Norleucine; Pbf: 2,2,4,6,7-pentamethyldihydrobenzofuran-5-sulfonyl; PBSS: Phosphate buffered saline NaCl; Poly(VDMO): poly(2-vinyl-4,4-dimethyl-5-oxazolone); SS: Sodium dodecyl sulfate solution with NaCl; T[4]: Spot for the thrombin specific probe 4; TFA-rhodamine-NHS-ester: 3′,6′-Di-trifluoroacetamido-spiro[phtalan-1,9′-xanthene]-5-carboxy succinimidyl ester and 3′,6′-Di-trifluoroacetamido-spiro[phtalan-1,9′-xanthene]-6-carboxy succinimidyl ester; TFA: Trifluoroacetic acid; THB: Thrombin buffer; THR: Thrombin; TIS: tri-isopropylsilane.

b) Synthesis of TFA-rhodamine-NHS ester

10.0 g 3-Aminophenol (0.091 mol, 3.3 eq.) were dissolved in 65.0 ml H₂SO₄ (95-97%). 5.76 g 1,2,4-Benzenetricarboxilic acid (0.027 mol, 1 eq.) were added and the stirred solution was warmed to 180° C. and kept at this temperature for 6 hours. After cooling to room temperature the reaction mixture was poured onto 70 g ice and stirred. The sulfuric acid was neutralized with solid sodium carbonate and 300 mL methanol were added to precipitate the inorganic salts. Inorganic salts were removed by filtration. The filter cake was washed with 200 mL methanol and the methanolic solutions were combined. After removal of the solvents, the residue was dissolved in methanol and adsorbed onto 30 g silica. The product was purified by suction column chromatography on 200 g silica using a step gradient using 3.5 L of acetonitrile/methanol (7:3) and then 1.5 L of acetonitrile/methanol/water/triethylamine (20:5:4:1). Evaporation of the solvents under reduced pressure yielded 6.5 g of a mixture of 3′,6′-Di-amino-spiro[phtalan-1,9′-xanthene]-5-carboxylic acid and 3′,6′-Di-amino-spiro[phtalan-1,9′-xanthene]-6-carboxylic acid (0.17 mol, 63%) as dark red crystalline solid. LC/MS characterization indicated about 90% purity. An analytical sample was obtained by recrystallization from acetone/water with a product purity>98%.

188 mg 3′,6′-Di-amino-spiro[phtalan-1,9′-xanthene]-5-carboxylic/6-carboxylic acid (0.5 mmol, 1 eq.) were co-evaporated three times with 1 mL dry pyridine and suspended in 4 mL dry pyridine. 190 mL trifluoroacetic acid anhydride (1.3 mmol, 2.6 eq) were added dropwise. The reaction mixture was stirred over night and the pyridine was removed under reduced pressure. The residue was dissolved in 2 mL CH₂Cl₂ and 403 mg N-hydroxysuccinimide (3.5 mmol, 7 eq.) and 477 mg 1-ethyl-3-(3′-dimethylaminopropyl)carbodiimide hydrochloride (2.5 mmol, 5 eq.) were added. The reaction mixture was stirred for 35 min and transferred to a separation funnel with 100 mL CH₂Cl₂ and 100 mL water. The organic phase was dried with sodium sulfate, filtered and the solvent was removed under reduced pressure. Column chromatography on 20 g silica using a step gradient of hexane/ethyl acetate from 75:25 to 45:55 afforded 46 mg TFA-rhodamine-NHS ester (0.07 mmol, 14% over 2 steps).

c) Synthesis of the rhodamine resin

Rink amide-Lys(Mtt)-Fmoc resin was prepared by condensation of Fmoc-Lys(Mtt) to Rink amide AM resin under standard HOBt/DICI coupling conditions. The substitution level of the resin was determined by Fmoc analysis (Bunin, B. A., The Combinatorial Index (Academic Press, San Diego; 1998)) to be 0.44 mmol/g. 900 mg of the Rink amide-Lys(Mtt)-Fmoc resin were placed in a reaction vessel of an Argonaut Quest 210 and washed twice with DMF (10 mL). The Fmoc protection group was removed by treatment with 20% (v/v) piperidine in DMF. The resin was washed with DMF (3×, 12 mL) and a solution of the TFA-rhodarnine-NHS ester (750 mg, 1.13 mmol) with HOBt (178 mg, 1.31 mmol) and DEEA (196 μl, 1.13 mmol) in DMF (4.5 mL) was added to the resin and agitated over night. The resin was washed three times with DMF (10 mL) and four times with CH₂Cl₂ (10 mL), filtered and blown dry with nitrogen. The coupling was quantitative as determined by LC-MS analysis.
d) Synthesis of the peptide part of 1-4 on the rhodamine scaffold

Rhodamine(TFA)-Lys(Mtt) Rink amide resin (250 mg) was placed in a reaction vessel of an Argonaut Quest 210 and hydrated with DMF (2×, 10 mL). The trifluoracetyl groups of the rhodamine were removed by treatment with concentrated aqueous ammonia (10 mL) for 4 h. The resin was washed five times with DMF (10 mL). Fmoc-Asp(O-t-Bu)—OH or Fmoc-Arg(Pbf)-OH was coupled to the resin using HATU and collidine in DMF (0.5 M Fmoc-amino acid, HATU and collidine, 12 mL, 24 h). Coupling of the arginine residues was repeated twice. The resin was washed with DMF (3×10 mL) and any remaining free rhodamine amino functions were acetylated overnight using acetic acid, DICI and 3-nitrotriazole (1 M each in DMF, 12 mL). Then the resin was washed as above. The coupling efficiency was determined by LC-MS to be 86% for the Fmoc-Asp(O-t-Bu) and 61% for the Fmoc-Arg(Pbf).

Following Fmoc-deprotection (20% piperidine in DMF, 2×5 mL) the next amino acid was coupled for 24 hours to the resin (0.3 M amino acid, DICI, HOBT, 3 mL each). This coupling and deprotection scheme was used to produce the sequences (nTPR)₂-rhodamine and (DEVD)₂-rhodamine (where n represents norleucine). The coupling yields were >90% as determined by Fmoc analysis (Bunin, B. A., The Combinatorial Index (Academic Press, San Diego; 1998)).

The final Fmoc group was removed from the peptide as above and the resin was washed with DMF (3×, 10 mL). The N-termini were acetylated using acetic acid, DICI and HOBt (0.3 M each in DMF, 3 mL, 1 h). Finally, the resin was washed three times with DMF (10 mL, 20 min) and four times with CH₂Cl₂ (10 mL, 20 min), filtered and blown dry with nitrogen.

e) Synthesis of the rhodamine substrates 1 and 3

50 mg (Ac-nTPR)₂-rhodamine-Lys(Mtt) or 50 mg (Ac-DEVD)₂-rhodamine-Lys(Mtt) resin were solvated with DMF (5 mL) and washed with CH₂Cl₂ (3×5 mL). The Mtt group was cleaved (dichloromethane, TFA and TIS at 94:1:5, 2 mL each, 2 min reaction time, four times) and the resin was washed with 0.2% (v/v) DIEA in DMF (3×5 mL, 20 min). The free lysine i-amino moiety was acetylated using acetic acid, HOBt and DICI in DMF (0.3 M each, 3 mL each, 1 h). The resin was washed three times with DMF (10 mL, 20 min) and four times with CH₂Cl₂ (10 mL, 20 min), filtered and blown dry with nitrogen.

The rhodamine-peptides were cleaved from the resin using 5 mL cleavage cocktail (TFA, water and TIS, 95: 2.5: 2.5) for 2 h and the solutions were concentrated under reduced pressure to 3 ml. A 1 mL aliquot of each product solution was precipitated into diethyl ether (40 mL) and centrifuiged (30 min, 4000×g, 4° C.). The precipitation of the (Ac-nTPR)₂-rhodamine-Lys(Ac) and (Ac-DEVD)₂-rhodamine-Lys(Ac) yielded 7.5 and 8.5 mg, respectively, corresponding to the yield of 65% for 3 and 75% for 1. The products were dissolved in 10% (v/v) DMF in H₂O. The correct mass of 3 and 1 was confrrmed using MALDI-TOF mass spectrometry: M(3)=1561.8, found: 1562.9([M+H]⁺); M(1)=1543.6, found: 1544.1 ([M+H]⁺), 1567.3 ([M+Na]⁺).

f) Synthesis of the PNA encoded rhodamine substrates 2 and 4

For the PNA encoded rhodamine substrates 10 mg of the (Ac-nTPR)₂-rhodamine-Lys(Mtt) or (Ac-DEVD)2-rhodamine-Lys(Mtt) resin were placed in an Applied Biosystems 2′-deoxyribo-oligonucleotide synthesis cartridge and a 50 mL syringe without plunger was attached. The resin was washed with CH₂Cl₂ (30 mL) using gravity flow. Afterwards 1% (v/v) TFA in CH₂Cl₂ (50 mL) were applied to remove the Mtt group from the resin. The resin was then washed with CH₂Cl₂ (20 mL), 0.2% (v/v) DIEA in DMF (30 mL) and dry DMF (50 mL). The syringe was detached and the cartridge was placed in an Applied Biosystems Expedite 8909 PNA synthesizer. The PNA was synthesized on a 2 μmol scale using standard ABI protocols.

After the PNA synthesis, the dry resin was transferred into 1.5 mL eppendorf tubes and the product was cleaved off the resin using 20% m-cresol in TFA (400 μl) for 3 hours. The product was precipitated into 1.5 mnL diethylether and pelleted by centrifugation (20,000×g, 20° C., 5 min). The supernatant was decanted and the pellet resuspended in 1.5 mL diethylether and centrifuged as above. After three extractions the pellet was dried at room temperature and dissolved in H₂O.

The correct mass of 4 and 2 was confirmed using MALDI-TOF mass spectrometry: M(4)=5553.7, found: 5553.3 ([M+H]⁺); M(2)=5596.4, found: 5595.5 ([M+H]⁺)

g) Synthesis of the 192 member PNA encoded protease substrate library

Rink amide-Lys(Mtt)-Fmoc resin was prepared by condensation of Fmoc-Lys(Mtt) to Rink amide AM resin under standard HOBt/DICI coupling conditions. The substitution level of the resin was determined by Fmoc analysis to be 0.25 mmol/g [Bunin, 1998 #55]. Rink amide-Lys(Mtt)-Fmoc resin (1 g) was placed into a reaction vessel of an Argonaut Quest 210 and washed twice with DMF (10 mL). The Fmoc protection group was removed by treatment with 20% (v/v) piperidine in DMF (5 mL, 10 min, 3×). The resin was washed with DMF (12 mL, 3×) and a solution of the TFA-rhodamine-NHS ester (600 mg, 0.9 mmol) and DIEA (173 μL, 1 mmol) in DMF (6 mL) was added to the resin and agitated over night. The resin was washed with DMF (10 mL, 10×) and with CH₂Cl₂ (10 mL, 4×), filtered and blown dry with nitrogen. The coupling was quantitative as determined by LC-MS analysis.

Rhodamine(TFA)-Lys(Mtt) Rink amide resin (1 g) was placed in a reaction vessel of an Argonaut Quest 210 and hydrated with DMF (10 mL, 15 min, 2×). The trifluoracetyl groups of rhodamine were removed by over night treatment with concentrated aqueous ammonia (10 mL). The resin was washed five times with DMF (10 mL). The resin was split into three aliquots of 333 mg and Fmoc-Asp(O-t-Bu)-OH or Fmoc-Arg(Pbf)-OH or Fmoc-Leu-OH was coupled to the resin using HATU and collidine in DMF (0.5 M Fmoc-amino acid, HATU and collidine, 12 mL, 24 h). The coupling of the arginine residues was repeated four times and the coupling of the aspartic and leucin was repeated two times. The resin was washed with DMF (10 mL, 3×) and any remaining free rhodamine amino functions were acetylated overnight using acetic acid, DICI and 3-nitrotriazole (1 M each in DMF, 12 mL each). The resin was washed as above. The coupling efficiency was determined by LC-MS analysis to be 87% for the Fmoc-Asp(O-t-Bu), 62% for the Fmoc-Arg(Pbf) and 80% for the Fmoc-Leu residue.

The loading of each resin was determined by Fmoc analysis and found to be 0.17 mmol/g for the Fmoc-Arg(Pb), 0.19 mmol/g for the Fmoc-Leu and 0.21 mmol/g for the Fmoc-Asp(O-t-Bu) resin, respectively. The amounts of the each resin used for the library synthesis reflected the differences in the previous coupling efficiency: 133 mg Fmoc-Arg(Pbf) resin, 120 mg Fmoc-Leu and 108 mg Fmoc-Asp(O-t-Bu) resin were each suspended in 2 mL DMF and split into four aliquots of 0.5 ml and transferred to new reaction vessels. the Fmoc protecting groups were removed (3 mL, 20% Piperidine in DMF, 10 min, 3×) and the resins washed (3 mL DMF, 5×). Each resin was modified with Alloc-Phe or Alloc-Val or Alloc-Pro or Alloc-Thr(t-Butyl) using HATU as coupling reagent (0.05 mmol Alloc-Amino acid, 0.05 mmol DIEA, 0.08 mmol collidine and 0.045 mmol HATU). Any remaining free amines remaining were capped (0.3 M acetic acid, DICI, HOBT, 3 mL each) for one hour and washed five times with DMF (5 mL) and washed with CH₂Cl₂ for 10 min (5 mL, 3×). A solution of 2% (v/v) TFA with 0.5% (v/v) TIS in CH₂Cl₂ (5 mL) was applied for 10 min, resulting in removal of the Mtt group from the resin. The treatment was repeated three times. The resin was washed with CH₂Cl₂ (5 mL, 3x), 2% (v/v) DIEA in DMF (5 mL, 3×) and dry DMF (5 mL, 4×). A Fmoc-AEEA-OH PNA spacer was coupled to the resin for two hours (0.04 mmol Fmoc-AEEA-OH PNA spacer monomer with 0.04 mmol DIEA, 0.06 mmol collidine and 0.035 mmol HATU in 0.5 mL DMF). The coupling step was repeated and any remaining free amino moieties were capped as above. The resin was washed with DMF (5 mL, 5×) and deprotected with 20% (v/v) piperidine in DMF (5 mL, 3 x, 10 min). The resin was washed with DMF (5 mL, 5×) and a second Fmoc-AEEA-OH PNA spacer was coupled to the resin and capped as above. The resin was Fmoc deprotected and washed with DMF as above. Fmoc-Lys (Boc) was coupled to the resin (0.04 mmol Fmoc-Lys(Boc), 0.04 mmol DIEA, 0.06 mmol collidine and 0.035 mmol HATU in 0.5 mL DMF) and capped as above. For the coupling of the first base of the first PNA codon the resin was deprotected and washed with DMF as above. The sequences of the PNA “codons” coupled to the resins are listed in FIG. 2B.

The bases of the PNA codons for the first and second amino acids were added by repetitive coupling of the corresponding PNA monomer, capping with acetic acid anhydride/collidine and Fmoc deprotection: 0.04 mmol of the corresponding PNA base monomer with 0.04 mmol DIEA, 0.06 mmol collidine and 0.035 mmol HATU in 0.5 mL DMF were added to the resin and the reaction mixture was shaken for one hour. The resin was washed with DMF (3 mL, 3×) and the coupling step was repeated. The resin was washed with DMF (5 mL, 5×), capped by treatment with acetic aced anhydride and collidine in DMF (0.04 mmol acetic acid anhydride, 0.06 mmol collidine in 0.5ml DMF, 5 min), washed with DMF as above and deprotected with 20% (v/v) piperidine in DMF (5 mL, 3×, 10 min). After washing with DMF (5 mL, 3×) the next three PNA bases were coupled to the resin as above (FIG. 2A). After the first codon was complete, the resins carrying the same second amino acids were unified and the PNA codons for the second amino acid were coupled to the resins (0.08 mmol of the corresponding PNA monomer, 0.08 mmol DIEA, 0.12 mmol collidine and 0.07 mmol HATU in 1 mL DMF). The coupling step was repeated. The resin was washed with DMF (10 mL, 5×), capped by treatment with acetic acid anhydride and collidine in DMF (0.08 mmol acetic acid anhydride, 0.12 mmol collidine in 1 mL DMF, 5 min), washed with DMF as above and deprotected with 20% (v/v) piperidine in DMF (10 mL, 3×, 10 min). After washing with DMF (10 mL, 3×) the residual PNA bases were coupled as above.

The final PNA base was left Fmoc protected. The resin was washed with CH₂Cl₂ (10 mL, 5×) and the alloc protection groups were removed by treatment with Pd(PPh₃)₄, Et₃SiH and acetic acid (0.02 mmol, 0.2 rnmol, 0.2 mmol, respectively, in 1 mL CH₂Cl₂, 2 h). The completion of the cleavage was monitored using MALDI-TOF mass spectroscopy. The resins were washed with CH₂Cl₂ (5 mL, 4×), DMF (5 mL, 4×), pooled, suspended in 4 mL DMF and aliquots of 1 mL were distributed to four new reaction vessels. Then the next alloc-amino acid (Asp or Arg or Thr or Pro) was coupled to the resin (0.11 mmol alloc amino acid, 0.11 mmol DIEA, 0.16 mmol collidine and 0.09 mmol HATU in 1.8 mL DMF) and any free amino moieties remaining were capped for one hour (0.3 M acetic acid, DICI, HOBT, 3 mL each). The resin was washed as above and the PNA codon for the amino acid was added to the resin. The resins were washed, pooled and aliquoted as above and the codon for the last amino acid was coupled using the same protocol as above. After the final Fmoc deprotection Fmoc-Lys(Boc) was coupled to the resins for two hours (0.11 mmol Fmoc-Lys(Boc), 0.11 mmol DIEA, 0.16 mmol collidine and 0.09 mmol HATU in 1.8 mL DMF). The resins were Fmoc deprotected as above and acetylated for 1 hour using acetic acid, HOBT and DICI (0.3 M each, 5 mL). The resins were washed with DMF (5 mL, 5×, 10 min), CH₂Cl₂ (10 mL, 4×, 10 min) and alloc deprotected as above. The corresponding Fmoc amino acid (Asp or Arg or Nle or Pro) was coupled to the resins, Fmoc deprotected, washed and acetylated (acetic acid, HOBT and DICI, 0.3 M each, 5 mL, 1 h). Final Fmoc analysis indicated an average yield of 50% for the amino acid coupling of the library. The resins were washed and combined.

The library was cleaved for one hour from the resin using a solution of TFA, m-Cresol and H₂O (80% (v/v), 19% (v/v), 1% (v/v), respectively, 5 mL). The cleavage procedure was repeated and the library was precipitated into 100 mL Et₂O and pelleted by centrifugation (20,000×g, 20° C., 10 min). The supernatant was decanted and the pellet resuspended in the same volume diethylether and centrifuiged as above. After four extractions the pellet was dried at room temperature and dissolved in 10 mL H₂O.

h) Enzymatic activity monitored using protease substrates 1 and 3

The substrates 1 and 3 were used at 250 μM. Thrombin was used at a concentration of 500 pM and caspase-3 was used at a concentration of 10 nM. For thrombin a buffer consisting of 50 mM Tris (pH 7.4),200 mM NaCl, 5 mM CaCI₂ and 0.01% (v/v) Tween-20 (THB) was used. The buffer for caspase-3 (CAB) consisted of 20 mM HEPES (pH 7.4), 100 mM NaCi, inM EDTA, 0.1% CHAPS,10% (w/v) sucrose and 10 mM DTT. 50 ll of the buffer containing the substrate at a concentration of 500 JIM were transferred into a well of a black 96-well Microfluor plate (Dynex Technologies, Chantilly, Va.). The reaction was initiated by the addition of 50 μL of the buffer containing the enzyme at a concentration of 1 nM for thrombin or 20 nM for caspase-3. The hydrolysis of rhodamine substrates was measured with a Spectramax Gemini XS spectrofluorimeter (Molecular devices, Sunnyvale, Calif.) thermostated at 37° C. using an excitation wavelength of 490 nm, an emission wavelength of 530 nm and a cutoff wavelength of 515 nm.

i) Single Substrate Kinetics

Thrombin was used at a final concentration of 1.25 nM and caspase-3 was used at a final concentration of 50 nM. The final concentration of the substrates ranged from 1 μM to 150 μM; the final concentration of DMF in the assay was less than 5%. 10 ,L of the buffer containing the substrate were transferred into a well of a black 384-well plate with clear bottom (Corning, N.Y.). The reaction was initiated by the addition of 10 μL of buffer containing the enzyme at a concentration of 2.5 nM for thrombin or 100 nM for caspase-3 and monitored by fluorescence as described above.

j) Determination of the Substrate Concentration using Total Hydrolysis

Serial dilutions of the substrates were performed in the corresponding buffers and thrombin or caspase-3 was added to a final concentration of 5 nM or 100 nM, respectively. The mixture was incubated overnight at room temperature. The endpoint fluorescence was measured as described above. The concentration of the rhodamine substrate for each dilution was determined by comparison of its fluorescence with the fluorescence of solutions containing known amounts of the rhodamine in the same buffer system.

Total hydrolysis of the 192 member library was performed in THB by sequential treatment with the following proteases: protease from Streptomycis Griseus, subtilisin Carlsbad and trypsin from bovine pancreas. Total hydrolysis was confirmed by MALDI-TOF analysis. The total concentration of the rhodamine library was determined by fluorescence measurement as described above.

k) Spatial Deconvolution of Single Protease Probes on Affymetrix Arrays

For single substrates the Affymetrix Geneflex array was used. The substrates 2 and 4 were diluted to 1 μM into THB containing with or without 100 nM caspase-3. The samples were incubated overnight at room temperature. The solutions were diluted with a modified PBS buffer, containing 250 mM NaCl (PBSS). The final substrate concentration ranged from 1 to 800 pM.

The Affymetrix GenFlex Arrays were hydrated by applying 180 μL CHB (100 mM MES, pH 6.5, 1 M NaCl) to the chips followed by incubation of the chips for 1 h at 45° C. in an Affymetrix hybridization oven. The CHB was removed and the chips were washed two times with PBSS. 6 μL solution of Affymetrix GeneFlex control probes was added to 180 μL of the diluted substrate solutions and the samples were applied to the GenFlex chips. The samples were hybridized for 4 h at 45° C. to the chips and the sample solutions were removed. The chips were washed three times with 180 μL PBSS and filled with 180 μL PBSS. The chips were read on an Affymetrix chip reader using the standard argon ion laser as light source and 530 nm as detection wavelength. The average intensity of 10 randomly picked border probes was used for normalization.

l) Limited Hydrolysis of the 192 Member PNA Encoded Protease Substrate Library

The 192 member PNA encoded library was diluted to a final concentration of 33 μM into 1 mL THB or CAB containing 3% (v/v) DMSO. Caspase-3 was used at a final concentration of 100 nM and the mixture was incubated at room temperature and the fluorescence was monitored over time until the desired percentage of hydrolysis was reached. The hydrolysis was monitored by fluorescence as described above. When the desired percentage of hydrolysis was reached, an aliquot of 200 ILL was removed and the enzymatic hydrolysis was quenched by adding 3 μL of a TFA/Water (1:5) solution. After the collection of all samples the solutions were diluted to a final concentration of 2 μM into PBSS with 3% (v/v) DMSO (50 μL final volume) and centrifuged (20.000×g, 4° C., 20 min).

m) Apoptotic vs Non Apoptotic Cell Lysates

Whole Jurkat cells (10⁷) were incubated for 4 hours with and without 100 ng/mL of a fas-activating antibody CH-1 1 (Kaminya Biomedical Co., Seattle, Wash.). The cells were washed twice with PBS and cytosolic lysates were prepared by treating the cells with 250 μl buffer containing 10 mM HEPES (pH 7.4),130 mM NaCl and 1% (v/v) triton X-100. The soluble cytosolic fraction was separated from the insoluble membrane fraction by centrifugation.

For single substrates 150 μL apoptotic or non apoptotic cell lysate diluted to a protein concentration of 1 mg/ml were mixed with 150 μL CAB containing 2 and 4 (2 μM each) and incubated at 37° C. Aliquots of 40 μL were withdrawn after 0, 1, 2, 3 and 6 hours. 25 μL of the aliquot were diluted into 75 μL PBS and the endpoint fluorescence was measured as described above. 10 μL of the aliquot were mixed with 1 μL of a TFA/H₂O (1:1) solution thereby quenching the enzymatic hydrolysis of the substrates. The quenched aliquots were placed on ice. After the collection of all time points 990 μL PBSS were added to the quenched aliquots and mixed. The samples were centrifuged (20.000×g, 20° C., 20 min) and the supernatant was applied to the printed oligonucleotide arrays.

For the 192 member library 100 μl undiluted lysates were added to 100 μl CAB containing 6% (v/v) DMSO and 66 μM library. The lysates were incubated at 37 ° C. until the desired hydrolysis (3.3% for lysates) was obtained. An aliquot of I100 μL was removed and the enzymatic hydrolysis was quenched by adding 1.5 μL of a TFA/Water (1:5) solution The solutions were diluted to a final concentration of 2 μM library into PBSS with 3% (v/v) DMSO (50 μL final volume) and centrifuged (20.000×g, 4° C., 20 min). The supernatants were applied to the printed oligonucleotide arrays.

n) Preparation of Amine Reactive Surfaces

Fluoropolymer masked glass slides (4×12 positive tone square-well array with well dimensions=3 mm x 3 mm; pitch adapted from standard 384-well plate spacing (Erie Scientific, Portsmouth, N.H.) were cleaned by agitation in an aqueous ammonia/ethanol solution (1:1 vol) for 1 h, followed by extensive rinsing with nanopure water and absolute ethanol. The glass slides were amino-functionalized by treating the freshly cleaned glass slides with a solution of 3-aminopropyltriethoxysilane under standard aqueous alkaline conditions (see, Silane Coupling Agents, 2nd edition. (Plenum: New York, 1991; and Tailoring Surfaces with Silanes. Chemtech 7, 766-778 (1977)). The glass slides were then immersed in a freshly prepared solution of poly(2-vinyl-4,4-dimethyl-5-oxazolone) (2.5 mg/ml) in NMP and 1% Et₃N for 24 h. The slides were washed several times alternately with DMF and chloroform, and finally with a 1,4-dioxane:toluene solution (1:1) before drying with a stream of nitrogen.

o) Printing of Oligonucleotide Arrays

For the experiments involving two single PNA encoded substrates two 3′-amino-modified oligonucleotides were printed in quadruplicate. The oligonucleotides for the caspase-3 feature ((Ac-DEVD)₂-Rh-PNAI) had the sequence 5′-CGC-TAG-ACT-ATC-GCC-C-3′ and the sequence used for the thrombin feature ((Ac-nTPR)₂-Rh-PNA2) was 5′-CAG-CGA-TGC-AGC-GTC-C-3′. For the on-chip kinetic experiments involving the 192 member PNA encoded library four 3′-amino-modified oligonucleotides were printed in triplicate using a spacing of 250 μm. The 3 ′-amino-modified oligonucleotides had the sequences 5′-GTC-CCA-CTG-CAT-GAA-GG-3′ (nTPR), 5′-GTC-CCA-GCT-CAT-GAA-GG-5′ (nTVR), 5′-GTC-CCA-GCT-TGT-TCA-GG-3′ (PRVR) and 5′-GTC-CCA-TCG-GTT-ATC-GG-3′ (RPFR). For the profiling experiments involving the 192 member PNA encoded library the 3′-amino-modified oligonucleotides were printed in three subarrays (8×8) according to the P1 position they encoded for.

The 3′-amino-modified oligonucleotides (50% DMSO, 75-250 μM oligonucleotide) were printed on amine reactive poly(VDMO) slides using an Omni Grid Accent contact printer (GeneMachnines, San Carlos, Calif.) at a spacing of 250 μm. The synthesis of the poly(VDMO) is described elsewhere (see, Tully, D. C., et al., Synthesis of reactive Poly(Vinyl Oxazolones) via nitroxide-mediated “living” free radical polymerization, Macromolecules (in print)). The printing was performed at 22° C. and 70% humidity using an SMP3 Stealth pin from TeleChem (Sunnyvale, Calif.). The slides were incubated overnight at 22° C. and 70% humidity followed by storage in a dessicator.

p) Postprocessing of Printed Oligonucleotide Arrays and Spatial Deconvolution of Single Protease Probes and the 192 Member PNA Encoded Library on Printed Oligonucleotide Arrays

The slides were submerged for three minutes into 92° C. hot SS solution (500 mM NaCl, 0.01% SDS), dip-rinsed three times in 250 mL nanopure water and dip-rinsed three times in a second batch of 250 mL nanopure water. The slides were dip-rinsed three times in ethanol and blown dry with nitrogen. The slides were deactivated by submersion in ethanolamine solution (0.5 M ethanolamine, pH 8.5) for 1 h. After washing with water and PBSS the slides were ready for sample application.

The samples were applied to the slides using a drop of 5 μl for each subarray on the slides with teflon masks. After application of all samples the slide was transferred into a 50 mL conical screw cap tube with water (500 μL). Alternatively, slides without teflon mask were incubated using a slide-holder that resembled a 384 well microtiter plate (see, Brinker et aL manuscript in preparation). In this case, 50 μl sample were applied to each well and the holder was closed with a tight-sealing lid. After incubation for four hours at 37° C. the slides were dip-rinsed in water (250 mL nanopure water, 3×) and centrifuged (1500×g, 2 min, 4° C.) and scanned on an Applied Precision 4500 scanner using the A488 filter set and an exposure time of 1.0 sec. ImaGene 4.2 software (BioDiscovery, Marina del Rey, Calif.) was used for data analysis. 2. Results and Discussion

One of the primary objectives of the present invention is to provide a microarray platform for the functional profiling of protease activity. The platform design comprises both a latent fluorophore that gives rise to a signal that is dependent on the presence of active proteases in addition to an encoding strategy that allows for the deconvolution of the signals using oligonucleotide microarrays. The approach enables either the simultaneous monitoring of different proteases in complex biological systems or the profiling of single proteases across many substrates.

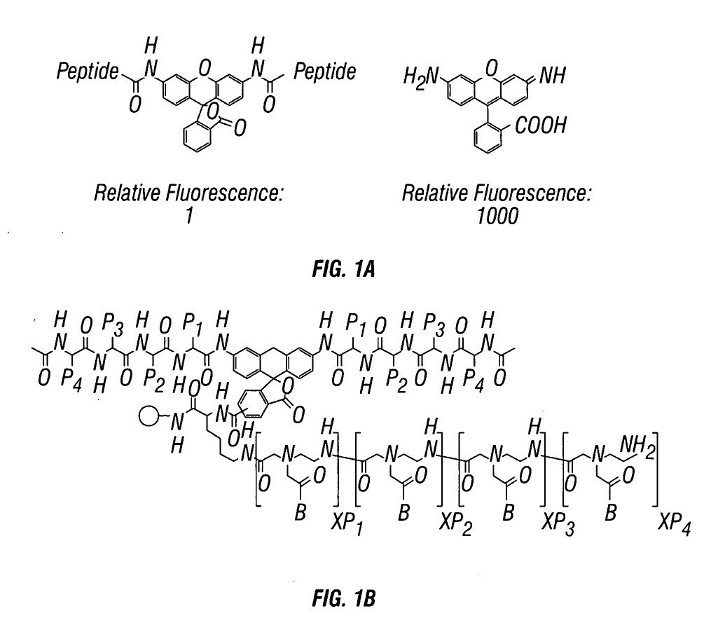

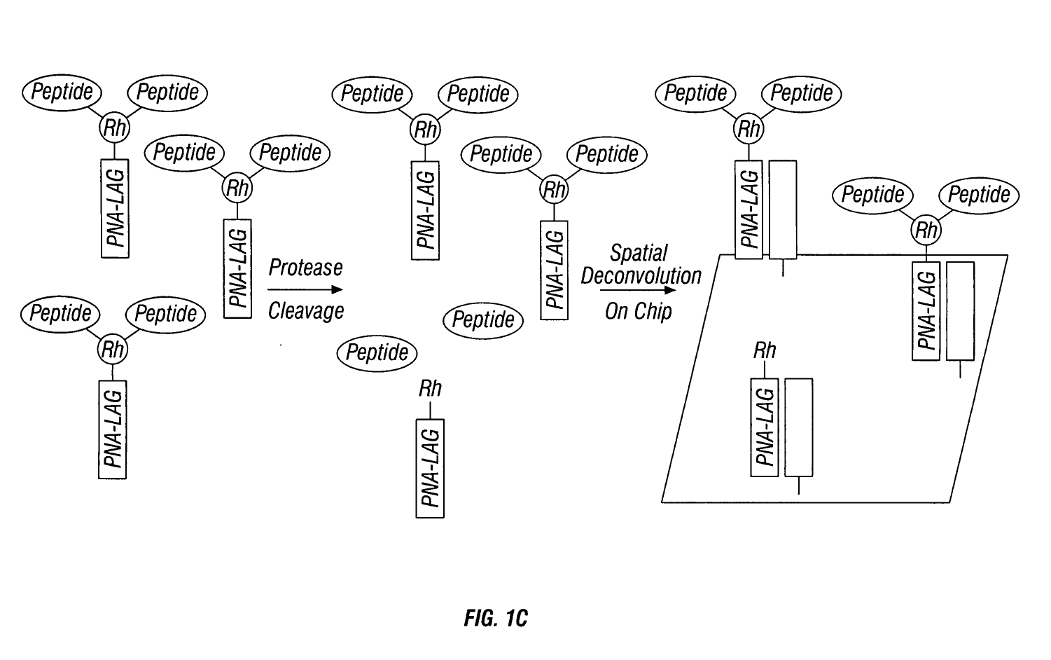

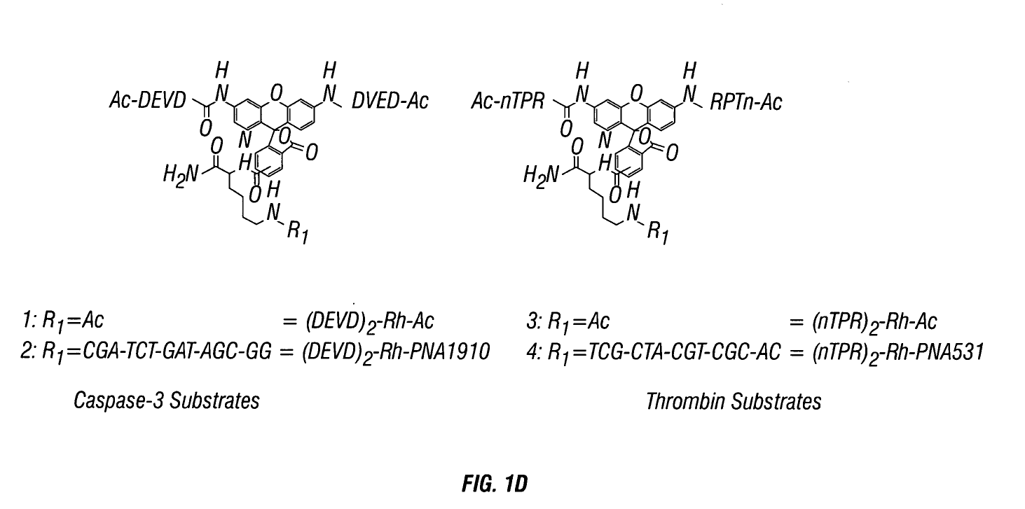

In one embodiment, the rhodamine scaffold was chosen as a bifunctional fluorophore upon which the substrates and substrate libraries would be constructed. Acylation of the rhodamine amino moieties diminishes its fluorescence approximately 1000 fold (FIG. 1A) (Leytus et al., Biochem. J., 209:299-307 (1983)). Peptides on the rhodamine scaffold have been shown to be accepted as substrates by serine and cysteine proteases (see, Assfalg-Machleidt et al., Biol. Chem. Hoppe Seyler, 373:433-440 (1992), and Leytus et al., Biochem. J., 215:253-260. (1983)). The enzymatic hydrolysis of the amide bond between the C-terminal carboxy residue of the peptide and the amino moiety of the rhodamine restores the original fluorescence allowing for direct and continuous monitoring of proteolytic activity. The rhodamine fluorophore exhibits some unique properties that make it particulary suited for this microarray application. First, the rhodamine possesses an absorbtion/emission spectrum which allows for the use of an argon-ion laser. Second, the fluorescence of the rhodamine is largely independent of the pH, allowing for the adjustment of the pH to the needs of a wide range of biological systems. Lastly, the starting material itself, TFA-rhodamine-NHS ester, is accessible in large quantities using inexpensive starting materials.

When PNA encoded rhodamine peptide substrates are incubated with an active protease, the protease recognizes its peptide substrate sequence and hydrolyzes the amide bond between rhodamine and peptide (FIG. 1C). The mixture of different probes containing hydrolyzed and unhydrolyzed probes is then put on an array consisting of spatially positioned oligonucleotides. The PNA portion of the probes hybridize with their antisense oligonucleotides, but only the probes containing rhodamines with free amino moieties give rise to fluorescence signals (FIG. 1C). Deconvolution of the fluorescence signal from multiple probes is therefore accomplished by encoding each amino acid of the peptides on the rhodamine scaffold in a PNA codon (FIG. 1B).

PNA is particularly suited for the hybridization to a DNA chip since the DNA-PNA interaction is much stronger than a corresponding DNA-DNA interaction. A mismatch by one base-pair in a PNA-DNA hybrid has much stronger effects, leading to less crosstalk between probes encoded by similar PNA sequences. Furthermore, the PNA encoding strategy is ideal for the library synthesis as it allows for the rapid generation of large libraries employing a combinatorial split-pool synthesis format. This strategy requires the use of orthogonal protecting groups for the extension of the peptide and PNA chains that in the present study is the alternating use of alloc protected amino acids and Fmoc protected PNA's monomers (FIG. 2A).

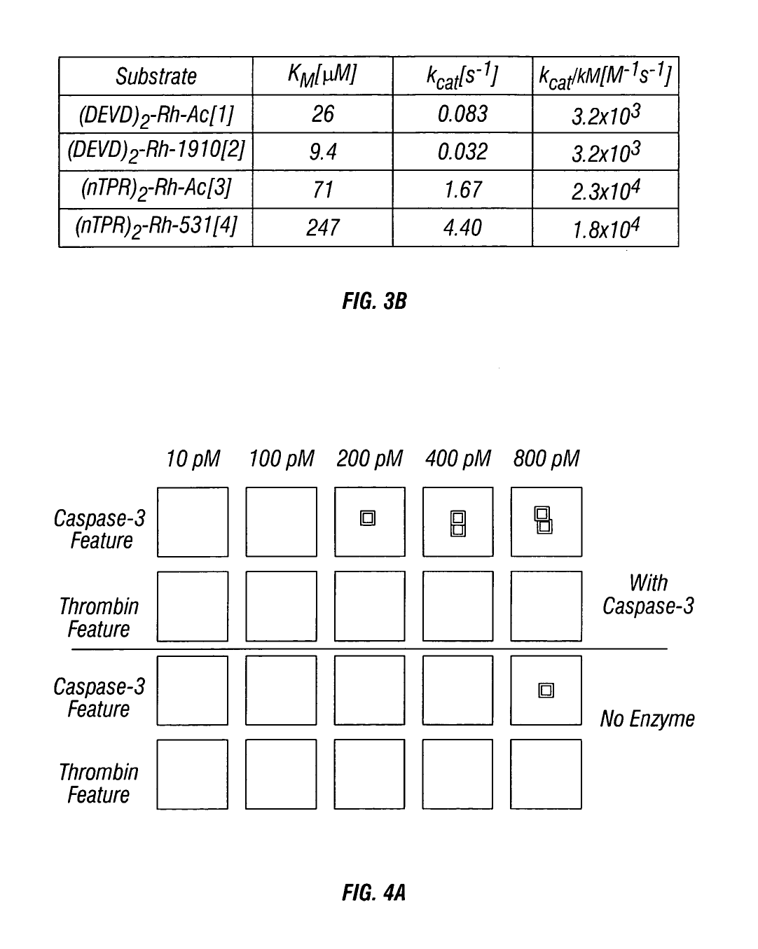

In order to evaluate the biological activity of the rhodamine peptide substrates two substrate pairs—rhodamine peptide substrate with and without PNA tag—for proteases whose substrate specificities are known were synthesized. The N-terminal to C-terminal sequences nTPR (where n represents norleucine) and DEVD, representing the preferred substrate sequences for the orthogonal proteases thrombin and caspase-3, respectively, were synthesized on the rhodamine scaffold (FIG. iD, 1-4) (Harris et al., Proc. Natl. Acad. Sci. USA, 97:7754-7759 (2000); and Cai et al., Bioorg. Med. Chem. Lett., 11, 39-42 (2001)). Both substrates without PNA tag are efficiently hydrolyzed by their corresponding enzymes (FIG. 3A). Upon incubation of the (DEVD)₂-rhodamine-Ac substrate with caspase-3 and incubation of the (nTPR)₂-rhodamine-Ac substrate with thrombin a linear increase in fluorescence indicating the enzymatic hydrolysis of the rhodamine substrates was observed. When incubated with the orthogonal enzyme, only a slight increase in fluorescence was observed (FIG. 3A) indicating the rhodamine substrated exhibit good substrate selectivity. Importantly, the Michaelis-Menten parameters and selectivity of PNA-tagged substrate were comparable to the substrates lacking the PNA tag (FIG. 3B). This is in agreement with previous findings that the PNA identifier tag did not interfere with the inhibitory activity of PNA-inhibitor adducts (see, Winssinger et al., Angewandte Chemie, International Edition, 40:3152-3155. (2001)).

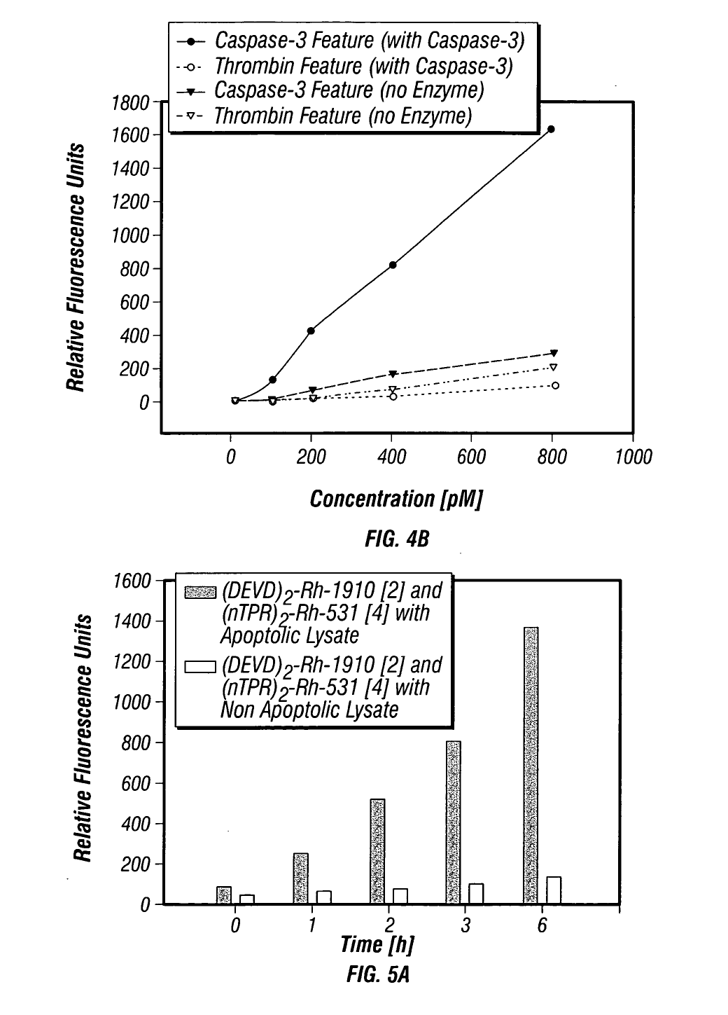

The issue of quantification of enzymatic activity by spatial deconvolution on a DNA microarray was then addressed. An equimolar mixture of the two single PNA encoded probes was incubated with or without caspase-3 and dilutions were loaded on an Affymetrix GenFlex chips (FIG. 4A). Only the caspase-3 feature of the chip showed a strong signal increase (FIG. 4A/B).

Next, the feasibility of PNA encoded rhodamine substrate libraries was evaluated. A 192 member tetrapeptide library was prepared that consisted of three different amino acids in the PI and four different amino acids in the P2-P4 positions. The PNA codons are non repetitive in order to avoid annealing between different PNA encoded probes or hairpin formation. In order to achieve a homogeneous annealing behavior, the selection of the codons was focused towards obtaining similar melting points of the PNA-oligonucleotide hybrids. The length of the codons itself was chosen as four bases encoding for the PI amino acid and three bases for P2-P4 amino acids, thereby weighting the oftentimes more important P1 site stronger than the less important P2-P4 sites during the hybridization process.

An important application of PNA encoded protease substrates libraries is to profile proteolytic activity in complex biological samples for the identification of therapeutic targets. A good model for this application would be the differential screening for caspase activation in apoptotic cell lysates in comparison to nonapoptotic cell lysates.

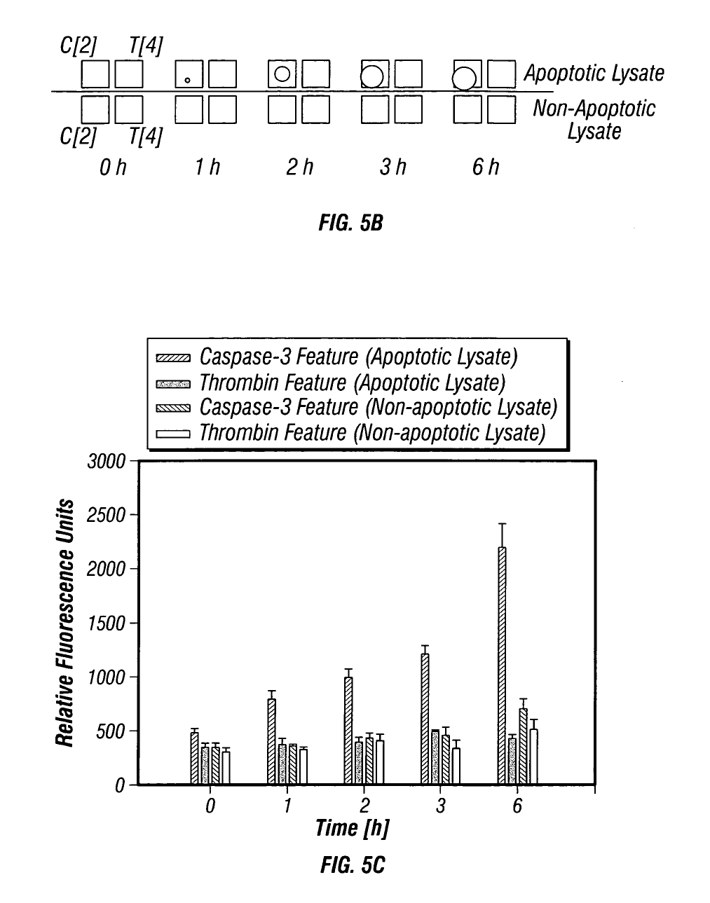

To test the robustness of the system, apoptotic and non apoptotic jurkat cell lysates were screened for caspase-3 activity. Upon incubation of the probes 2 and 4 with the lysates a strong increase in total fluorescence over time was obtained only in the sample with the apoptotic lysate. The fluorescence of the sample containing the nonapoptotic lysate remained nearly constant (FIG. 5A). Spatial deconvolution of these samples on oligonucleotide microarrays showed a fluorescence increase only for the caspase-3 feature indicating the apoptotic activation of the caspase-3 (FIGS. 5B and 5C). Apoptosis does not manifest itself as an enhanced expression of caspase-3, but rather as an increase in enzymatic activity which is due to a proteolytic conversion of the caspase-3 zymogen. Changes in proteolytic activities that are based on posttranslational modification cannot-be monitored by expression profiling experiments using DNA arrays emphasizing the importance of measuring protein function rather than simply mRNA message or protein levels.

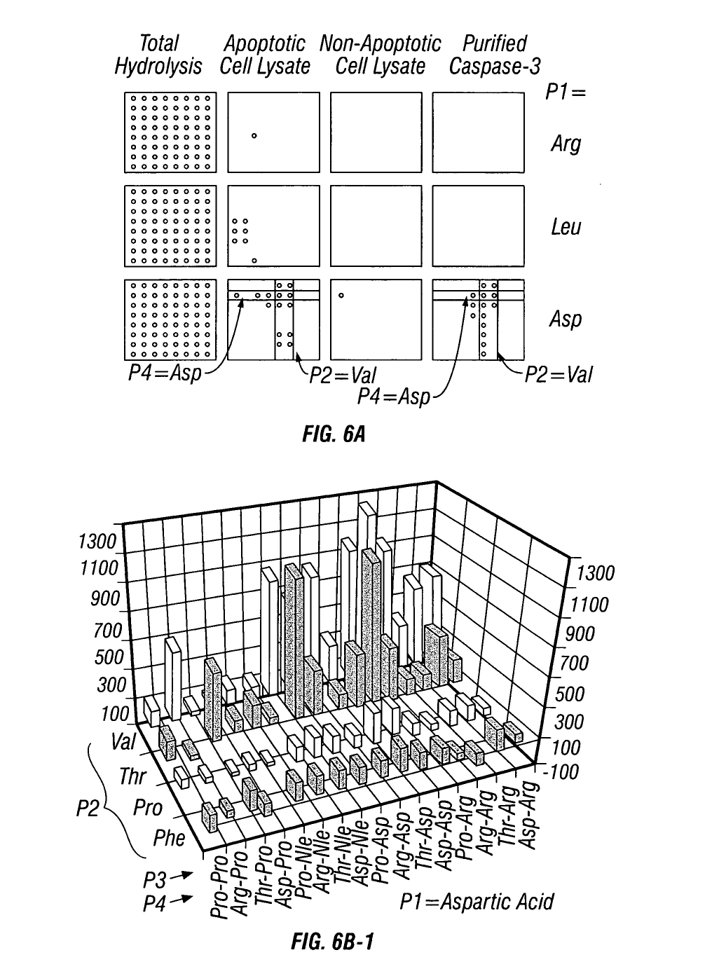

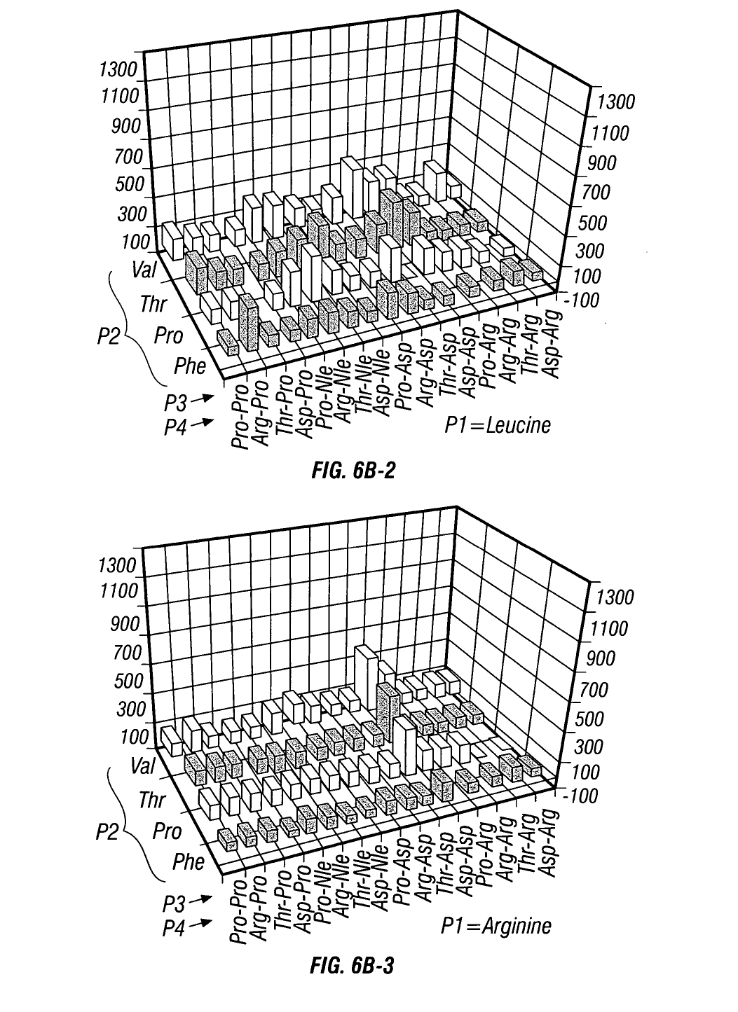

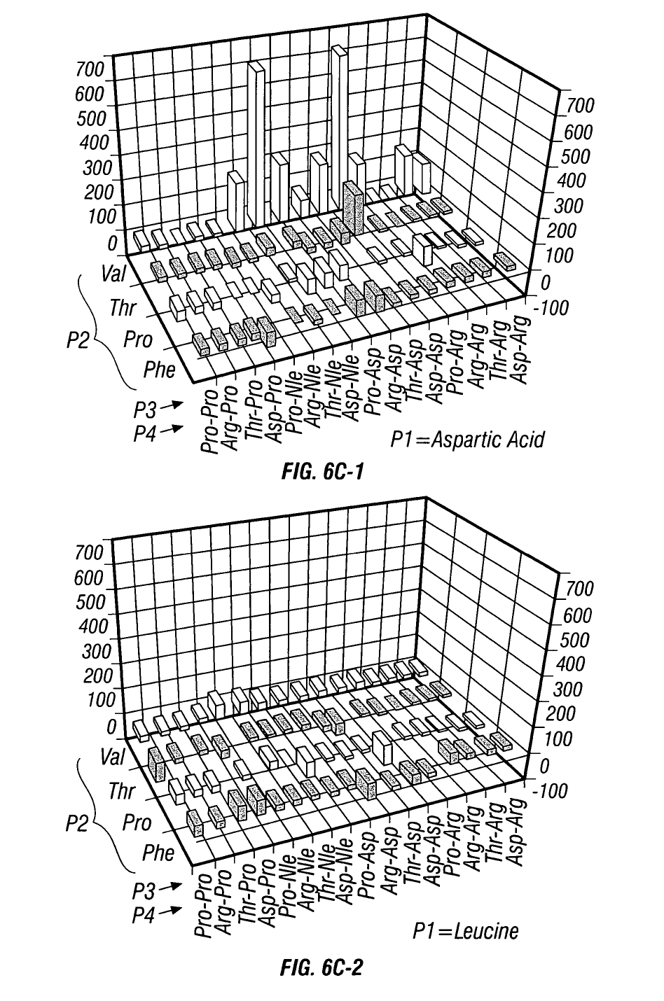

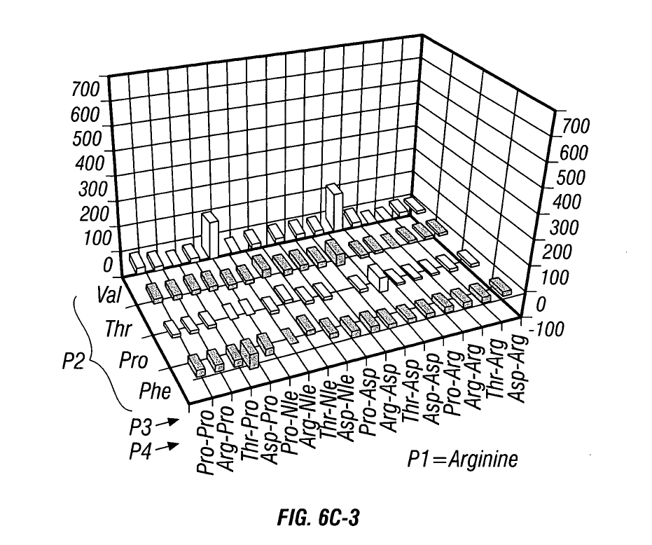

Next, the issue of using the substrate library to generate substrate specific profiles in complex biological samples was addressed. The 192 member PNA encoded substrate library was treated either with apoptotic or nonapoptotic cell lysate and deconvoluted on the combinatorial oligonucleotide arrays. The most intense signals observed in the apoptotic lysate are substrate sequences with a valine in P2 and an aspartic acid in P4 position, similar to those found for the purified caspase-3. The activity fingerprint of caspase-3 can be clearly seen in the differential profile of the apoptotic and nonapoptotic lysate (FIG. 6A). Therefore, it is possible to use the methods of the present invention for the differential screening for proteolytic activities in complex biological samples in a microarrays format using substrate specificity profiles.

In addition to the foregoing, a synthesis method has been developed for a bifunctional rhodamine fluorophore that allows for the parallel synthesis of the peptidic substrate and its encoding PNA tag using the efficient split-pool method. A major advantage of the presented strategy is that the proteolytic cleavage of the substrates takes place in solution, thereby excluding potentially detrimental solid surface effects while simultaneously providing greater flexibility in the conditions used (pH, concentration, buffer and temperature). The feasibility of PNA encoded rhodamine substrate libraries was demonstrated by the synthesis of a 192 member PNA encoded protease substrate library. Differences in cleavage rates between optimal and poorer substrates can be readily visualized on a chip indicating that on-chip kinetics are feasible. The robustness of the methods of the present invention was demonstrated by measuring the caspase-3 activation during apoptosis in crude cell lysates, suggesting the feasibility of PNA-encoded protease substrates for the screening of drug targets in clinical samples. The limited volume of the clinical samples which is currently a mayor obstacle in direct screening of these samples is overcome by the methods of the present invention. The presented microarray based methods are valuable in gaining a better understanding of the complex proteolytic events involved in the regulation of cellular processes and pathogenesis of many diseases.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

1. A compound comprising:
(a) a fluorogenic moiety;
(b) an organic moiety covalently attached to said fluorogenic moiety, wherein said organic moiety comprises a cleavage recognition site for an enzyme; and
(c) a petido nucleic acid (PNA) identifier tag covalently attached to said fluorogenic moiety, wherein said PNA identifier tag identifies said organic moiety.

2. The compound in accordance with claim 1, wherein when said organic moiety is covalently attached to said fluorogenic moiety, the fluorescence of said fluorogenic moiety is quenched.

3. The compound in accordance with claim 1, wherein said fluorogenic moiety is a rhodamine moiety.

4. The compound in accordance with claim 3, wherein said rhodamine moiety is a rhodamine NHS ester.

5. The compound in accordance with claim 1, wherein said fluorogenic moiety is a coumarin moiety.

6. The compound in accordance with claim 1, wherein said coumarin moiety is a member selected from the group consisting of 7-amino-4-methylcoumarin (AMC), 7-amino-4-trifluoromethylcoumarin (AFC), 7-amino-4-chloromethylcoumarin (CMAC) and 7-amino-4-carbamoylmethylcoumarin (ACC).

7. The compound of claim 1, wherein said PNA identifier tag is from about 3 to about 50 nucleotides in length.

8. The compound of claim 1, wherein said PNA identifier tag is from about 6 to about 20 nucleotides in length.

9. The compound of claim 1, wherein said PNA identifier tag is from about 12 to about 14 nucleotides in length.

10. The compound in accordance with claim 1, wherein said organic moiety is a member selected from the group consisting of an amino acid, a polypeptide sequence, a nucleotide sequence, a lipid, a carbohydrate and a small organic molecule.

11. The compound in accordance with claim 1, wherein said fluorogenic moiety is a fluorescence donor moiety.

12. The compound in accordance with claim 11, wherein said compound further comprises a fluorescence acceptor moiety.

13. The compound in accordance with claim 12, wherein said fluorescence acceptor moiety is covalently attached to said fluorescence donor moiety through said organic moiety.

14. The compound in accordance with claim 1, wherein said enzyme is a nucleophilic enzyme.

15. The compound in accordance with claim 14, wherein said nucleophilic enzyme is a hydrolase.

16. The compound in accordance with claim 15, wherein said hydrolase is a protease.

17. The compound in accordance with claim 16, wherein said protease is a member selected from the group consisting of aspartic proteases, cysteine proteases, metalloproteases, threonine proteases and serine proteases.

18. The compound in accordance with claim 15, wherein said hydrolase is a lipase.

19. The compound in accordance with claim 15, wherein said hydrolase is a phosphatase.

20. The compound in accordance with claim 1, wherein said organic moiety is an amino acid.

21. The compound in accordance with claim 1, wherein said organic moiety is a polypeptide sequence.

22. The compound in accordance with claim 1, wherein said organic moiety is a lipid.

23. The compound in accordance with claim 1, wherein said organic moiety is a small organic molecule.

24. The compound in accordance with claim 23, wherein said small organic molecule comprises an amide bond.

25. The compound in accordance with claim 23, wherein said small organic molecule comprises a phosphate ester.

26. The compound in accordance with claim 21, wherein said polypeptide sequence is covalently attached to said fluorogenic moiety through an amide bond, wherein said amide bond is formed between a carboxylic acid moiety of the carboxy terminus of said polypeptide sequence and an amine of said fluorogenic moiety.

27. The compound in accordance with claim 1, wherein said compound further comprises a second organic moiety.

28. The compound in accordance with claim 27, wherein said second organic moiety is a member selected from the group consisting of an amino acid, a polypeptide sequence, a nucleotide sequence, a lipid, a carbohydrate and a small organic molecule.

29. The compound in accordance with claim 28, wherein said second organic moiety is a polypeptide sequence.

30. The compound in accordance with claim 29, wherein said first organic moiety and said second organic moiety are the same.

31. The compound in accordance with claim 1, wherein said organic moiety further comprises a quencher.

32. The compound in accordance with claim 1, wherein said compound has the following structure: 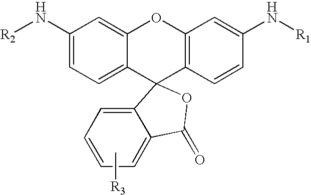
wherein:
R¹ and R² are independently selected from the group consisting of an amino acid, a polypeptide sequence, a nucleotide sequence, a lipid, a carbohydrate and a small organic molecule; and
R³ is a PNA identifier tag.

33. The compound in accordance with claim 32, wherein R′ and R² are both polypeptide sequences, said polypeptide sequences having the following structure:
-C(O)-AA¹-AA²-(AAⁱ)_{J-2}
wherein:
AA¹-AA-(AAⁱ)_{J-2} is a polypeptide sequence, wherein each of AA¹ through AAⁱ is an amino acid residue which is a member independently selected from the group of natural amino acid residues, unnatural amino acid residues and modified amino acid residues;
J denotes the number of amino acid residues forming said polypeptide sequence and is a member selected from the group consisting of the numbers from 2 to 10, such that J-2 is the number of amino acid residues in the polypeptide sequence exclusive of AA¹-AA²;
i denotes the position of said amino acid residue relevant to AA¹ and when J is greater than 2, i is a member selected from the group consisting of the numbers from 3 to 10; and
R³ is a PNA identifier tag.

34. The compound in accordance with claim 32, wherein said PNA identifier tag is from about 3 to about 50 nucleotides in length.

35. The compound in accordance with claim 32, wherein said PNA identifier tag is from about 6 to about 20 nucleotides in length.

36. The compound in accordance with claim 32, wherein said PNA identifier tag is from about 12 to about 14 nucleotides in length.

37. The compound in accordance with claim 1, wherein said compound has the following structure: 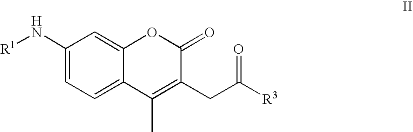
wherein:
R¹ is a member selected from the group consisting of an amino acid, a polypeptide sequence, a nucleotide sequence, a lipid, a carbohydrate and a small organic molecule; and
R³is a PNA identifier tag.

38. The compound in accordance with claim 13, wherein said compound has the following structure: 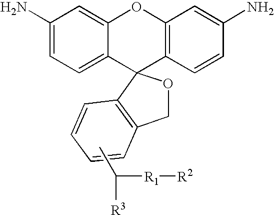
wherein:
R¹ is a member selected from the group consisting of an amino acid, a polypeptide sequence, a nucleotide sequence, a lipid, a carbohydrate and a small organic molecule;
R² is a fluorescence acceptor moiety; and
R³is a PNA identifier tag.

39. The compound in accordance with claim 13, wherein said compound has the following structure: 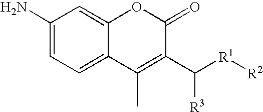
wherein:
R¹ is a member selected from the group consisting of an amino acid, a polypeptide sequence, a nucleotide sequence, a lipid, a carbohydrate and a small organic molecule;
R² is a fluorescence acceptor moiety; and
R³ is a PNA identifier tag.

40. A compound comprising:
(a) a fluorescence donor moiety;
(b) a fluorescence acceptor moiety;
(c) an organic moiety comprising a cleavage recognition site for an enzyme, wherein said fluorescence donor moiety is covalently attached to said fluorescence acceptor moiety through said organic moiety; and
(d) a petido nucleic acid (PNA) identifier tag covalently attached to said fluorescence donor moiety, wherein said PNA identifier tag identifies said organic moiety.

41. A method for assaying for the presence of an enzymatically active enzyme in a sample, said method comprising:
(a) contacting said sample with a compound in accordance with claim 1 under conditions such that if said enzymatically active enzyme is present in said sample, at least a portion of said organic moiety is cleaved from said fluorogenic moiety of said compound, thereby producing a fluorescent compound having said PNA identifier tag covalently attached thereto;
(b) hybridizing said fluorescent compound to an array of oligonucleotides; and
(c) detecting said fluorescent compound that hybridizes to said array of oligonucleotides, wherein detection of said fluorescent compound indicates the presence of said enzymatically active enzyme in said sample.

42. The method in accordance with claim 41, wherein said enzymatically enzyme is a protease.

43. The method in accordance with claim 41, wherein said protease is a member selected from the group consisting of aspartic proteases, cysteine proteases, metalloproteases, threonine proteases and serine proteases.

44. The method in accordance with claim 41, wherein said proteases is a protease of a microorganism.

45. The method in accordance with claim 44, wherein said microorganism is a member selected from the group consisting of bacteria, fungi, yeast, viruses and protozoa.

46. The method in accordance with claim 41, wherein said sample is a clinical sample.

47. The method in accordance with claim 41, further comprising (d) quantifying said fluorescent compound, thereby quantifying said protease.

48. The method in accordance with claim 41, wherein said compound in accordance with claim 1 has the following structure: 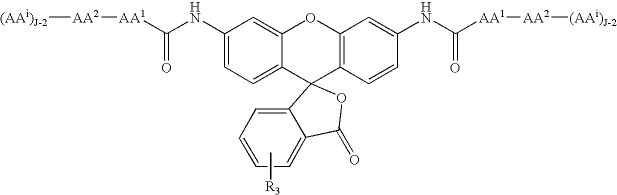
wherein:
each AA¹-AA²-(AAⁱ)_{J-2} is a polypeptide sequence, wherein each of AA¹ through AAⁱ is an amino acid residue which is a member independently selected from the group of natural amino acid residues, unnatural amino acid residues and modified amino acid residues;
J denotes the number of amino acid residues forming said polypeptide sequence and is a member selected from the group consisting of the numbers from 2 to 10, such that J-2 is the number of amino acid residues in the polypeptide sequence exclusive of AA¹-AA²; and
i denotes the position of said amino acid residue relevant to AA¹ and when J is greater than 2, i is a member selected from the group consisting of the numbers from 3 to 10.

49. A method for detecting activation of a biological pathway by assaying for the presence of an enzymatically active enzyme in a sample, said method comprising:
(a) contacting said sample with a compound in accordance with claim 1 under conditions such that if said enzymatically active enzyme is present in said sample, at least a portion of said organic moiety is cleaved from said fluorogenic moiety of said compound, thereby producing a fluorescent compound having said PNA identifier tag covalently attached thereto;
(b) hybridizing said fluorescent compound to an array of oligonucleotides; and
(c) detecting said fluorescent compound that hybridizes to said array of oligonucleotides, wherein detection of said fluorescent compound indicates the presence of said enzymatically active enzyme in said sample, and wherein the presence of said enzymatically active enzyme in said sample indicates activation of said biological pathway.

50. The method in accordance with claim 49, wherein said biological pathway is a member selected from the group consisting of apoptosis, hemostasis, blood coagulation, immunological processes, ubiquitination, proteolysis, cell division, cell growth, signaling cascades, processing of antigens for presentation on the surface of cells, differentiation pathways, survival pathways, neurotransmitter release, cell migration, cell adhesion, complement activation, stress-response pathways and metabolic pathways.

51. The method in accordance with claim 50, wherein said biological pathway is apoptosis.

52. The method in accordance with claim 49, wherein said enzymatically active enzyme is a protease.

53. The method in accordance with claim 52, wherein said protease is a member selected from the group consisting of aspartic proteases, cysteine proteases, metalloproteases, threonine proteases and serine proteases.

54. The method in accordance with claim 49, wherein said sample is a cell, tissue or organ lysate.

55. The method in accordance with claim 49, wherein said sample is a biological fluid selected from the group consisting of sputum, blood, blood cells, tissue or fine needle biopsy samples, urine, peritoneal fluid and pleural fluid.

56. A library of fluorogenic enzyme substrates comprising at least a first fluorogenic enzyme substrate and a second fluorogenic enzyme substrates, wherein said first and second fluorogenic enzyme substrates comprise: (a) a fluorogenic moiety; (b) an organic moiety covalently attached to said fluorogenic moiety, wherein said organic moiety comprises a cleavage recognition site for an enzyme; and (c) a petido nucleic acid (PNA) identifier tag covalently attached to said fluorogenic moiety, wherein said PNA identifier tag identifies said organic moiety.

57. A library of fluorogenic polypeptides comprising at least a first fluorogenic polypeptide and a second fluorogenic polypeptide, wherein said first and second fluorogenic polypeptides have the following structure: 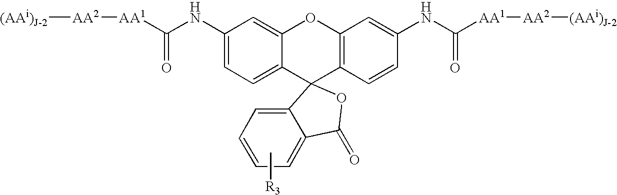
wherein:
each AA¹-AA²-(AAⁱ)_{J-2} is a polypeptide sequence, wherein each of AA¹ through AAⁱ is an amino acid residue which is a member independently selected from the group of natural amino acid residues, unnatural amino acid residues and modified amino acid residues;
J denotes the number of amino acid residues forming said polypeptide sequence and is a member selected from the group consisting of the numbers from 2 to 10, such that J-2 is the number of amino acid residues in the polypeptide sequence exclusive of AA¹-AA²;
i denotes the position of said amino acid residue relevant to AA¹ and when J is greater than 2, i is a member selected from the group consisting of the numbers from 3 to 10.; and
R³ is a PNA identifier tag.

58. The library in accordance with claim 57, wherein the polypeptide sequences of said first fluorogenic polypeptide are different from the polypeptide sequence of said second fluorogenic polypeptide.

59. The library in accordance with claim 57, wherein an amino acid residue selected from the group consisting of AA¹, AA², AAⁱ and combinations thereof of the polypeptide sequences of said first polypeptide is a different amino acid residue than an amino acid residue at a corresponding position relative to AA¹ of the polypeptide sequences of said second polypeptide.

60. The library in accordance with claim 57, wherein AA¹ of the polypeptide sequences of said first polypeptide and AA¹ of the polypeptide sequences of said second polypeptide are identical.

61. The library in accordance with claim 57, wherein AA¹ of the polypeptide sequence of said first polypeptide and AA¹ of the polypeptide sequence of said second polypeptide are different.

62. The library in accordance with claim 57, wherein said library comprises at least 10 fluorogenic polypeptides having different polypeptide sequences.

63. The library in accordance with claim 62, wherein AA¹ is a different amino acid residue in each of said different polypeptide sequences.

64. The library in accordance with claim 57, wherein said library comprises at least 100 fluorogenic polypeptides having different polypeptide sequences.

65. The library in accordance with claim 57, wherein said library comprises at least 10³ fluorogenic polypeptides having different polypeptide sequences.

66. The library in accordance with claim 57, wherein said library comprises at least 10⁴ fluorogenic polypeptides having different polypeptide sequences.

67. A method for determining a polypeptide sequence specificity profile of an enzymatically active protease, said method comprising:
(a) contacting said protease with a library of fluorogenic polypeptides in accordance with claim 57, wherein said polypeptide sequences are selectively cleaved by said protease, thereby producing a fluorescent compound having said PNA identifier tag covalently attached thereto;
(b) hybridizing said fluorescent compound to an array of oligonucleotides;
(c) detecting said fluorescent compound that hybridizes to said array of oligonucleotides; and
(d) determining the sequence of said polypeptide sequences, thereby identifying said polypeptide sequence specificity profile of said protease.

68. The method in accordance with claim 67, further comprising (e) quantifying said fluorescent compound, thereby quantifying said protease.


SEQUENCE LISTING (155 sequences)

SEQ ID NO: 1 - DNA, 14 bp, Artificial Sequence
  peptide nucleic acid (PNA) encoding caspase-3 rhodamine peptidyl protease substrate (DEVD)-2-Rh-PNA1910
ngatctgatagcgg

SEQ ID NO: 2 - DNA, 14 bp, Artificial Sequence
  peptide nucleic acid (PNA) encoding thrombin rhodamine peptidyl protease substrate (nTPR)-2-Rh-PNA531
ncgctacgtcgcag

SEQ ID NO: 3 - protein, 4 aa, Artificial Sequence
  rhodamine protease substrate library peptide
DVPP

SEQ ID NO: 4 - protein, 4 aa, Artificial Sequence
  rhodamine protease substrate library peptide
DVRP

SEQ ID NO: 5 - protein, 4 aa, Artificial Sequence
  rhodamine protease substrate library peptide
DVTP

SEQ ID NO: 6 - protein, 4 aa, Artificial Sequence
  rhodamine protease substrate library peptide
DVDP

SEQ ID NO: 7 - protein, 4 aa, Artificial Sequence
  rhodamine protease substrate library peptide
DVPD

SEQ ID NO: 8 - protein, 4 aa, Artificial Sequence
  rhodamine protease substrate library peptide
DVRD

SEQ ID NO: 9 - protein, 4 aa, Artificial Sequence
  rhodamine protease substrate library peptide
DVTD

SEQ ID NO: 10 - protein, 4 aa, Artificial Sequence
  rhodamine protease substrate library peptide
DVDD

SEQ ID NO: 11 - protein, 4 aa, Artificial Sequence
  rhodamine protease substrate library peptide
DVPR

SEQ ID NO: 12 - protein, 4 aa, Artificial Sequence
  rhodamine protease substrate library peptide
DVRR

SEQ ID NO: 13 - protein, 4 aa, Artificial Sequence
  rhodamine protease substrate library peptide
DVTR

SEQ ID NO: 14 - protein, 4 aa, Artificial Sequence
  rhodamine protease substrate library peptide
DVDR

SEQ ID NO: 15 - protein, 4 aa, Artificial Sequence
  rhodamine protease substrate library peptide
DTPP

SEQ ID NO: 16 - protein, 4 aa, Artificial Sequence
  rhodamine protease substrate library peptide
DTRP

SEQ ID NO: 17 - protein, 4 aa, Artificial Sequence
  rhodamine protease substrate library peptide
DTTP

SEQ ID NO: 18 - protein, 4 aa, Artificial Sequence
  rhodamine protease substrate library peptide
DTDP

SEQ ID NO: 19 - protein, 4 aa, Artificial Sequence
  rhodamine protease substrate library peptide
DTPD

SEQ ID NO: 20 - protein, 4 aa, Artificial Sequence
  rhodamine protease substrate library peptide
DTRD

SEQ ID NO: 21 - protein, 4 aa, Artificial Sequence
  rhodamine protease substrate library peptide
DTTD

SEQ ID NO: 22 - protein, 4 aa, Artificial Sequence
  rhodamine protease substrate library peptide
DTDD

SEQ ID NO: 23 - protein, 4 aa, Artificial Sequence
  rhodamine protease substrate library peptide
DTPR

SEQ ID NO: 24 - protein, 4 aa, Artificial Sequence
  rhodamine protease substrate library peptide
DTRR

SEQ ID NO: 25 - protein, 4 aa, Artificial Sequence
  rhodamine protease substrate library peptide
DTTR

SEQ ID NO: 26 - protein, 4 aa, Artificial Sequence
  rhodamine protease substrate library peptide
DTDR

SEQ ID NO: 27 - protein, 4 aa, Artificial Sequence
  rhodamine protease substrate library peptide
DPPP

SEQ ID NO: 28 - protein, 4 aa, Artificial Sequence
  rhodamine protease substrate library peptide
DPRP

SEQ ID NO: 29 - protein, 4 aa, Artificial Sequence
  rhodamine protease substrate library peptide
DPTP

SEQ ID NO: 30 - protein, 4 aa, Artificial Sequence
  rhodamine protease substrate library peptide
DPDP

SEQ ID NO: 31 - protein, 4 aa, Artificial Sequence
  rhodamine protease substrate library peptide
DPPD

SEQ ID NO: 32 - protein, 4 aa, Artificial Sequence
  rhodamine protease substrate library peptide
DPRD

SEQ ID NO: 33 - protein, 4 aa, Artificial Sequence
  rhodamine protease substrate library peptide
DPTD

SEQ ID NO: 34 - protein, 4 aa, Artificial Sequence
  rhodamine protease substrate library peptide
DPDD

SEQ ID NO: 35 - protein, 4 aa, Artificial Sequence
  rhodamine protease substrate library peptide
DPPR

SEQ ID NO: 36 - protein, 4 aa, Artificial Sequence
  rhodamine protease substrate library peptide
DPRR

SEQ ID NO: 37 - protein, 4 aa, Artificial Sequence
  rhodamine protease substrate library peptide
DPTR

SEQ ID NO: 38 - protein, 4 aa, Artificial Sequence
  rhodamine protease substrate library peptide
DPDR

SEQ ID NO: 39 - protein, 4 aa, Artificial Sequence
  rhodamine protease substrate library peptide
DFPP

SEQ ID NO: 40 - protein, 4 aa, Artificial Sequence
  rhodamine protease substrate library peptide
DFRP

SEQ ID NO: 41 - protein, 4 aa, Artificial Sequence
  rhodamine protease substrate library peptide
DFTP

SEQ ID NO: 42 - protein, 4 aa, Artificial Sequence
  rhodamine protease substrate library peptide
DFDP

SEQ ID NO: 43 - protein, 4 aa, Artificial Sequence
  rhodamine protease substrate library peptide
DPPD

SEQ ID NO: 44 - protein, 4 aa, Artificial Sequence
  rhodamine protease substrate library peptide
DFRD

SEQ ID NO: 45 - protein, 4 aa, Artificial Sequence
  rhodamine protease substrate library peptide
DFTD

SEQ ID NO: 46 - protein, 4 aa, Artificial Sequence
  rhodamine protease substrate library peptide
DFDD

SEQ ID NO: 47 - protein, 4 aa, Artificial Sequence
  rhodamine protease substrate library peptide
DFPR

SEQ ID NO: 48 - protein, 4 aa, Artificial Sequence
  rhodamine protease substrate library peptide
DFRR

SEQ ID NO: 49 - protein, 4 aa, Artificial Sequence
  rhodamine protease substrate library peptide
DFTR

SEQ ID NO: 50 - protein, 4 aa, Artificial Sequence
  rhodamine protease substrate library peptide
DFDR

SEQ ID NO: 51 - protein, 4 aa, Artificial Sequence
  rhodamine protease substrate library peptide
LVPP

SEQ ID NO: 52 - protein, 4 aa, Artificial Sequence
  rhodamine protease substrate library peptide
LVRP

SEQ ID NO: 53 - protein, 4 aa, Artificial Sequence
  rhodamine protease substrate library peptide
LVTP

SEQ ID NO: 54 - protein, 4 aa, Artificial Sequence
  rhodamine protease substrate library peptide
LVDP

SEQ ID NO: 55 - protein, 4 aa, Artificial Sequence
  rhodamine protease substrate library peptide
LVPD

SEQ ID NO: 56 - protein, 4 aa, Artificial Sequence
  rhodamine protease substrate library peptide
LVRD

SEQ ID NO: 57 - protein, 4 aa, Artificial Sequence
  rhodamine protease substrate library peptide
LVTD

SEQ ID NO: 58 - protein, 4 aa, Artificial Sequence
  rhodamine protease substrate library peptide
LVDD

SEQ ID NO: 59 - protein, 4 aa, Artificial Sequence
  rhodamine protease substrate library peptide
LVPR

SEQ ID NO: 60 - protein, 4 aa, Artificial Sequence
  rhodamine protease substrate library peptide
LVRR

SEQ ID NO: 61 - protein, 4 aa, Artificial Sequence
  rhodamine protease substrate library peptide
LVTR

SEQ ID NO: 62 - protein, 4 aa, Artificial Sequence
  rhodamine protease substrate library peptide
LVDR

SEQ ID NO: 63 - protein, 4 aa, Artificial Sequence
  rhodamine protease substrate library peptide
LTPP

SEQ ID NO: 64 - protein, 4 aa, Artificial Sequence
  rhodamine protease substrate library peptide
LTRP

SEQ ID NO: 65 - protein, 4 aa, Artificial Sequence
  rhodamine protease substrate library peptide
LTTP

SEQ ID NO: 66 - protein, 4 aa, Artificial Sequence
  rhodamine protease substrate library peptide
LTDP

SEQ ID NO: 67 - protein, 4 aa, Artificial Sequence
  rhodamine protease substrate library peptide
LTPD

SEQ ID NO: 68 - protein, 4 aa, Artificial Sequence
  rhodamine protease substrate library peptide
LTRD

SEQ ID NO: 69 - protein, 4 aa, Artificial Sequence
  rhodamine protease substrate library peptide
LTTD

SEQ ID NO: 70 - protein, 4 aa, Artificial Sequence
  rhodamine protease substrate library peptide
LTDD

SEQ ID NO: 71 - protein, 4 aa, Artificial Sequence
  rhodamine protease substrate library peptide
LTPR

SEQ ID NO: 72 - protein, 4 aa, Artificial Sequence
  rhodamine protease substrate library peptide
LTRR

SEQ ID NO: 73 - protein, 4 aa, Artificial Sequence
  rhodamine protease substrate library peptide
LTTR

SEQ ID NO: 74 - protein, 4 aa, Artificial Sequence
  rhodamine protease substrate library peptide
LTDR

SEQ ID NO: 75 - protein, 4 aa, Artificial Sequence
  rhodamine protease substrate library peptide
LPPP

SEQ ID NO: 76 - protein, 4 aa, Artificial Sequence
  rhodamine protease substrate library peptide
LPRP

SEQ ID NO: 77 - protein, 4 aa, Artificial Sequence
  rhodamine protease substrate library peptide
LPTP

SEQ ID NO: 78 - protein, 4 aa, Artificial Sequence
  rhodamine protease substrate library peptide
LPDP

SEQ ID NO: 79 - protein, 4 aa, Artificial Sequence
  rhodamine protease substrate library peptide
LPPD

SEQ ID NO: 80 - protein, 4 aa, Artificial Sequence
  rhodamine protease substrate library peptide
LPRD

SEQ ID NO: 81 - protein, 4 aa, Artificial Sequence
  rhodamine protease substrate library peptide
LPTD

SEQ ID NO: 82 - protein, 4 aa, Artificial Sequence
  rhodamine protease substrate library peptide
LPDD

SEQ ID NO: 83 - protein, 4 aa, Artificial Sequence
  rhodamine protease substrate library peptide
LPPR

SEQ ID NO: 84 - protein, 4 aa, Artificial Sequence
  rhodamine protease substrate library peptide
LPRR

SEQ ID NO: 85 - protein, 4 aa, Artificial Sequence
  rhodamine protease substrate library peptide
LPTR

SEQ ID NO: 86 - protein, 4 aa, Artificial Sequence
  rhodamine protease substrate library peptide
LPDR

SEQ ID NO: 87 - protein, 4 aa, Artificial Sequence
  rhodamine protease substrate library peptide
LFPP

SEQ ID NO: 88 - protein, 4 aa, Artificial Sequence
  rhodamine protease substrate library peptide
LFRP

SEQ ID NO: 89 - protein, 4 aa, Artificial Sequence
  rhodamine protease substrate library peptide
LFTP

SEQ ID NO: 90 - protein, 4 aa, Artificial Sequence
  rhodamine protease substrate library peptide
LFDP

SEQ ID NO: 91 - protein, 4 aa, Artificial Sequence
  rhodamine protease substrate library peptide
LPPD

SEQ ID NO: 92 - protein, 4 aa, Artificial Sequence
  rhodamine protease substrate library peptide
LFRD

SEQ ID NO: 93 - protein, 4 aa, Artificial Sequence
  rhodamine protease substrate library peptide
LFTD

SEQ ID NO: 94 - protein, 4 aa, Artificial Sequence
  rhodamine protease substrate library peptide
LFDD

SEQ ID NO: 95 - protein, 4 aa, Artificial Sequence
  rhodamine protease substrate library peptide
LFPR

SEQ ID NO: 96 - protein, 4 aa, Artificial Sequence
  rhodamine protease substrate library peptide
LFRR

SEQ ID NO: 97 - protein, 4 aa, Artificial Sequence
  rhodamine protease substrate library peptide
LFTR

SEQ ID NO: 98 - protein, 4 aa, Artificial Sequence
  rhodamine protease substrate library peptide
LFDR

SEQ ID NO: 99 - protein, 4 aa, Artificial Sequence
  rhodamine protease substrate library peptide
RVTP

SEQ ID NO: 100 - protein, 4 aa, Artificial Sequence
  rhodamine protease substrate library peptide
RVPP

SEQ ID NO: 101 - protein, 4 aa, Artificial Sequence
  rhodamine protease substrate library peptide
RVDP

SEQ ID NO: 102 - protein, 4 aa, Artificial Sequence
  rhodamine protease substrate library peptide
RVRP

SEQ ID NO: 103 - protein, 4 aa, Artificial Sequence
  rhodamine protease substrate library peptide
RVTD

SEQ ID NO: 104 - protein, 4 aa, Artificial Sequence
  rhodamine protease substrate library peptide
RVPD

SEQ ID NO: 105 - protein, 4 aa, Artificial Sequence
  rhodamine protease substrate library peptide
RVDD

SEQ ID NO: 106 - protein, 4 aa, Artificial Sequence
  rhodamine protease substrate library peptide
RVRD

SEQ ID NO: 107 - protein, 4 aa, Artificial Sequence
  rhodamine protease substrate library peptide
RVTR

SEQ ID NO: 108 - protein, 4 aa, Artificial Sequence
  rhodamine protease substrate library peptide
RVPR

SEQ ID NO: 109 - protein, 4 aa, Artificial Sequence
  rhodamine protease substrate library peptide
RVDR

SEQ ID NO: 110 - protein, 4 aa, Artificial Sequence
  rhodamine protease substrate library peptide
RVRR

SEQ ID NO: 111 - protein, 4 aa, Artificial Sequence
  rhodamine protease substrate library peptide
RTTP

SEQ ID NO: 112 - protein, 4 aa, Artificial Sequence
  rhodamine protease substrate library peptide
RTPP

SEQ ID NO: 113 - protein, 4 aa, Artificial Sequence
  rhodamine protease substrate library peptide
RTDP

SEQ ID NO: 114 - protein, 4 aa, Artificial Sequence
  rhodamine protease substrate library peptide
RTRP

SEQ ID NO: 115 - protein, 4 aa, Artificial Sequence
  rhodamine protease substrate library peptide
RTTD

SEQ ID NO: 116 - protein, 4 aa, Artificial Sequence
  rhodamine protease substrate library peptide
RTPD

SEQ ID NO: 117 - protein, 4 aa, Artificial Sequence
  rhodamine protease substrate library peptide
RTDD

SEQ ID NO: 118 - protein, 4 aa, Artificial Sequence
  rhodamine protease substrate library peptide
RTRD

SEQ ID NO: 119 - protein, 4 aa, Artificial Sequence
  rhodamine protease substrate library peptide
RTTR

SEQ ID NO: 120 - protein, 4 aa, Artificial Sequence
  rhodamine protease substrate library peptide
RTPR

SEQ ID NO: 121 - protein, 4 aa, Artificial Sequence
  rhodamine protease substrate library peptide
RTDR

SEQ ID NO: 122 - protein, 4 aa, Artificial Sequence
  rhodamine protease substrate library peptide
RTRR

SEQ ID NO: 123 - protein, 4 aa, Artificial Sequence
  rhodamine protease substrate library peptide
RPTP

SEQ ID NO: 124 - protein, 4 aa, Artificial Sequence
  rhodamine protease substrate library peptide
RPPP

SEQ ID NO: 125 - protein, 4 aa, Artificial Sequence
  rhodamine protease substrate library peptide
RPDP

SEQ ID NO: 126 - protein, 4 aa, Artificial Sequence
  rhodamine protease substrate library peptide
RPRP

SEQ ID NO: 127 - protein, 4 aa, Artificial Sequence
  rhodamine protease substrate library peptide
RPTD

SEQ ID NO: 128 - protein, 4 aa, Artificial Sequence
  rhodamine protease substrate library peptide
RPPD

SEQ ID NO: 129 - protein, 4 aa, Artificial Sequence
  rhodamine protease substrate library peptide
RPDD

SEQ ID NO: 130 - protein, 4 aa, Artificial Sequence
  rhodamine protease substrate library peptide
RPRD

SEQ ID NO: 131 - protein, 4 aa, Artificial Sequence
  rhodamine protease substrate library peptide
RPTR

SEQ ID NO: 132 - protein, 4 aa, Artificial Sequence
  rhodamine protease substrate library peptide
RPPR

SEQ ID NO: 133 - protein, 4 aa, Artificial Sequence
  rhodamine protease substrate library peptide
RPDR

SEQ ID NO: 134 - protein, 4 aa, Artificial Sequence
  rhodamine protease substrate library peptide
RPRR

SEQ ID NO: 135 - protein, 4 aa, Artificial Sequence
  rhodamine protease substrate library peptide
RFTP

SEQ ID NO: 136 - protein, 4 aa, Artificial Sequence
  rhodamine protease substrate library peptide
RFPP

SEQ ID NO: 137 - protein, 4 aa, Artificial Sequence
  rhodamine protease substrate library peptide
RFDP

SEQ ID NO: 138 - protein, 4 aa, Artificial Sequence
  rhodamine protease substrate library peptide
RFRP

SEQ ID NO: 139 - protein, 4 aa, Artificial Sequence
  rhodamine protease substrate library peptide
RFTD

SEQ ID NO: 140 - protein, 4 aa, Artificial Sequence
  rhodamine protease substrate library peptide
RFPD

SEQ ID NO: 141 - protein, 4 aa, Artificial Sequence
  rhodamine protease substrate library peptide
RFDD

SEQ ID NO: 142 - protein, 4 aa, Artificial Sequence
  rhodamine protease substrate library peptide
RFRD

SEQ ID NO: 143 - protein, 4 aa, Artificial Sequence
  rhodamine protease substrate library peptide
RFTR

SEQ ID NO: 144 - protein, 4 aa, Artificial Sequence
  rhodamine protease substrate library peptide
RFPR

SEQ ID NO: 145 - protein, 4 aa, Artificial Sequence
  rhodamine protease substrate library peptide
RFDR

SEQ ID NO: 146 - protein, 4 aa, Artificial Sequence
  rhodamine protease substrate library peptide
RFRR

SEQ ID NO: 147 - DNA, 16 bp, Artificial Sequence
  peptide nucleic acid (PNA) 3′-amino modified oligonucleotide for caspase-3
cgctagactatcgccn

SEQ ID NO: 148 - DNA, 16 bp, Artificial Sequence
  peptide nucleic acid (PNA) 3′-amino modified oligonucleotide for thrombin
cagcgatgcagcgtcn

SEQ ID NO: 149 - DNA, 17 bp, Artificial Sequence
  peptide nucleic acid (PNA) 3′-amino modified oligonucleotide for PNA encoded library
gtcccactgcatgaagn

SEQ ID NO: 150 - DNA, 17 bp, Artificial Sequence
  peptide nucleic acid (PNA) 3′-amino modified oligonucleotide for PNA encoded library
gtcccagctcatgaagn

SEQ ID NO: 151 - DNA, 17 bp, Artificial Sequence
  peptide nucleic acid (PNA) 3′-amino modified oligonucleotide for PNA encoded library
gtcccagcttgttcagn

SEQ ID NO: 152 - protein, 4 aa, Artificial Sequence
  protease substrate library peptide
PRVR

SEQ ID NO: 153 - DNA, 17 bp, Artificial Sequence
  peptide nucleic acid (PNA) 3′-amino modified oligonucleotide for PNA encoded library
gtcccatcggttatcgn

SEQ ID NO: 154 - protein, 4 aa, Artificial Sequence
  protease substrate library peptide
RPFR

SEQ ID NO: 155 - protein, 4 aa, Artificial Sequence
  caspase-3 preferred protease substrate library peptide
DEVD